(19) United States
(12) Patent Application Publication
(10) Pub. No.: US 2024/0299292 A1
(43) Pub. Date: Sep. 12, 2024

(54) STABLE SEMI-SOLID CHEWABLE GEL COMPOSITIONS AND METHODS OF MAKING AND USING THEREOF

(71) Applicant: Seattle Gummy Company, Renton, WA (US)

(72) Inventors: Feng WAN, Sammamish, WA (US); William Brenden Carlson, Shoreline, WA (US)

(73) Assignee: Seattle Gummy Company, Renton, WA (US)

(21) Appl. No.: 18/039,272

(22) PCT Filed: Dec. 1, 2021
(86) PCT No.: PCT/US2021/061451

§ 371 Date: May 29, 2023

(51) Int. Cl. A61K 9/00; A61K 9/16; A61K 45/06

(52) U.S. Cl. CPC A61K 9/0056; A61K 9/1623; A61K 45/06

(57) ABSTRACT

A semi-solid chewable gel composition, comprising a gelling composition in a sufficient amount to provide a cohesive gelled product, a binding composition comprising a sugar, a sugar alcohol, or a combination thereof, wherein the sugar comprises psicose (allulose), sorbose, tagatose, D-maltose (1,4-diglucose), trehalose, isomaltulose, or a combination thereof, and a water-soluble polymer stabilizer, wherein the polymer stabilizer comprises a polymer of monosaccharide monomers selected from glucose, mannose, galactose, arabinose, rhamnose, xylose, galacturonate, glucuronate, N-acetylglucosamine, or a combination thereof, and wherein the polymer comprises from about 5 to about 500 monosaccharide monomers, wherein the semi-solid chewable gel composition is substantially free of glucose, sucrose, and fructose.

### CROSS-REFERENCES TO RELATED APPLICATIONS

This application claims the benefit of priority from, and hereby incorporates by reference the entire disclosure, co-pending US Provisional Applications for (1) Patent Serial No. 63/119,657, filed Dec. 1, 2020; (2) Patent Serial No. 63/119,661, filed Dec. 1, 2020; (3) Patent Serial No. 63/119,658, filed Dec. 1, 2020; and (4) and Patent Serial No. 63/119,660, filed Dec. 1, 2020.

### TECHNICAL FIELD

This application relates to semi-solid edible or chewable gel compositions with one or more bioactive incorporated therein.

### BACKGROUND

Unless otherwise indicated herein, the materials described in this section are not prior art to the claims in this application and are not admitted being prior art by inclusion in this section.

Pharmaceuticals are available in a variety of dosage forms for treating the diseases. Dosages that are formulated to take orally including tablets, capsules, soft-gels, powders, chewable tablets, and liquid suspensions.

Tablets, capsules and soft gels are difficult for individuals who have difficulties swallowing pills. This problem is magnified when the medications need to be taken 2-4 times per day to provide the desired therapeutic effect. Moreover, the need for a source of water or other liquid to assist with swallowing solid dosage forms can complicate administration.

Powders are often difficult to administer and chewable tablets can be hard to chew especially for seniors and young children. In addition, powders and chewable tablets often have an unpleasant after-taste.

Liquid suspensions or solutions are sometimes used as an alternative to solid oral dosage forms. However, the dosing with liquid dosage forms is not precise, which can lead to the administration of too little or too much medications. In addition, liquid dosage forms are messy and often have a bitter taste, which could impact person compliance.

Semi-solid chewable (gummy) composition could deliver medications and bioactive with an easier consumption profile. However, conventional gummy formulations are often packed with sugar and as a result having a high in glycemic index, making them unhealthy and potentially dangerous for diabetic patients.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides semi-solid chewable composition. In one embodiment, the semi-solid chewable gel composition comprises a gelling composition in a sufficient amount to provide a cohesive gelled product, a binding composition comprising a sugar alcohol, a saccharide, or a combination thereof, and a water-soluble polymer stabilizer, wherein the semi-solid chewable gel composition is substantially free of glucose, fructose, and sucrose. Example saccharides include monosaccharide, di-saccharide, tri-saccharide, or a combination thereof.

In one embodiment, the binding composition may include at least 2 binding agents selected from saccharides or sugar alcohols.

In one embodiment, the binding composition comprises a low GI (glycemic index) saccharide including without limitation mono-, di- or tri-saccharide. In one embodiment, the low GI monosaccharide comprises a stereoisomer of D-fructose, D-glucose, or D-galactose. In one embodiment, the low GI monosaccharide comprises L-fructose, L-glucose, L-galactose, allulose, sorbose, tagatose, or a combination thereof. In one embodiment, the low GI disaccharide comprises an isomer of D-maltose (1,4-diglucose) or an isomer of D-sucrose (1,2-fructose glucose). In one embodiment, the low GI disaccharide comprises trehalose, isomaltulose, or a combination thereof. In one embodiment, the low GI tri-saccharide comprises raffinose.

In one embodiment, the binding composition may include allulose by a weight of not less than 30%, 40%, 50%, 55%, 60% or 70%. In one embodiment, the binding composition may include allulose by a weight from about 50% to about 60%.

In one embodiment, the binding composition may include trehalose by a weight of not less than 15%, 20%, 22%, 22.5%, 25%, 30%, 40%, 50%, 55%, or 60%. In one embodiment, the binding composition may include trehalose by a weight from about 20% to about 30%.

In one embodiment, the binding composition may include isomaltulose by a weight of not less than 5%, 10%, 15%, 20%, 22%, 22.5%, 25%, 30%, 40%, 50%, 55%, or 60%. In one embodiment, the binding composition may include isomaltulose by a weight from about 10% to about 30%.

In one embodiment, the binding composition may include trehalose and isomaltulose. The a ratio may be from about 3:1 to about 1:3. In one embodiment, the binding composition may include trehalose and isomaltulose in a ratio of 3:1, 2:1, 1:1, 1:2, 2:1, or 3:1.

In one embodiment, the binding composition comprises trehalose, isomaltulose, and allulose. In one embodiment, the binding composition consists essentially of trehalose, isomaltulose and a third low GI sugar selected from L-fructose, L-glucose, L-galactose, allulose, Sorbose, Tagatose, or a combination thereof.

In one embodiment, the binding composition comprises tagatose, allulose, or a combination thereof. In one embodiment, the binding composition comprises tagatose, isomaltulose, or a combination thereof. In one embodiment, the binding composition comprises allulose, trehalose, isomaltulose, or a combination thereof.

In one embodiment, the binding composition consists essentially of sugar alcohols. In one embodiment, the binding composition comprises mannitol, sorbitol, xylitol, lactitol, isomalt, maltitol, hydrogenated starch hydrolysates (HSH), glycerol, erythritol, or a combination thereof.

In one embodiment, the binding composition comprises mannitol, maltitol, isomalt, or a combination thereof. In one embodiment, the binding composition comprises mannitol, sorbitol, or a combination thereof. In one embodiment, the binding composition comprises mannitol, sorbitol, erythritol or a combination thereof. In one embodiment, the binding composition comprises mannitol, sorbitol, isomalt, or a combination thereof. In one embodiment, the binding composition comprises mannitol, maltitol, sorbitol, or a combination thereof. In one embodiment, the binding composition comprises mannitol, maltitol, xylitol, or a combination thereof. In one embodiment, the binding composition comprises mannitol, xylitol, isomalt, or a combination thereof. In one embodiment, the binding composition comprises mannitol, xylitol, sorbitol or a combination thereof. In one embodiment, the binding composition may comprise maltitol, sorbitol or a combination thereof. The ratio of maltitol and sorbitol may be from about 1:1 to about 5:3.

In one embodiment, the binding composition comprises mannitol, sorbitol, isomalt, resistant starch or a combination thereof. In one embodiment, the binding composition comprises mannitol, maltitol, sorbitol, maltodextrin or a combination thereof. In one embodiment, the binding composition comprises xylitol. In one embodiment, the binding composition comprises erythritol.

In one embodiment, the binding composition may comprise allulose, maltitol, or a combination thereof. The ratio of allulose and maltitol may be from about 1:1 to about 2:1, about 10:7, or about 10:6. In one embodiment, the binding composition may comprise allulose, sorbitol or a combination thereof. The ratio of allulose and sorbitol may be from about 10:8 to about 2:1. In one embodiment, the binding composition may comprise allulose, xylitol or a combination thereof. The ratio of allulose and xylitol may be from about 10:8 to 2:1. In one embodiment, the binding composition may comprise maltitol, xylitol or a combination thereof. The ratio of maltitol and xylitol may be from about 8:10 to 3:2. In one embodiment, the binding composition may comprise allulose, erythritol or a combination thereof. The ratio of allulose and erythritol may be from about 1:1 to 2:1. In one embodiment, the binding composition consists essentially of maltitol, tagatose, or a combination thereof. In one embodiment, the binding composition consists essentially of isomalt, allulose, or a combination

In one embodiment, the binding composition comprises maltitol, sorbitol, allulose, or a combination thereof. In one embodiment, the binding composition comprises maltitol, tagatose, or a combination thereof. In one embodiment, the binding composition comprises allulose, tagatose, maltitol, isomalt, or a combination thereof. In one embodiment, the binding composition comprises isomalt, allulose, or a combination thereof. In one embodiment, the binding composition comprises isomalt, sorbitol, allulose, or a combination thereof. In one embodiment, the binding composition comprises allulose, xylitol, or a combination thereof. In one embodiment, the binding composition comprises allulose, maltitol, or a combination thereof.

In one embodiment, the binding composition consists essentially of trehalose, xylitol, sorbitol or a combination thereof. In one embodiment, the binding composition consists essentially of mannitol, isomaltulose, xylitol, sorbitol or a combination thereof. In one embodiment, the binding composition consists essentially of maltitol, sorbitol, allulose, or a combination thereof. In one embodiment, the inding composition consists essentially of allulose, tagatose, maltitol, isomalt, or a combination thereof. thereof. In one embodiment, the binding composition consists essentially of isomalt, sorbitol, allulose, or a combination thereof.

In one embodiment, the binding composition consists essentially of mannitol, N-acetylglucosamine, isomalt, or a combination thereof. In one embodiment, the binding composition comprises maltitol, allulose, resistant starch or a combination thereof. In one embodiment, the binding composition comprises isomalt, allulose, resistant maltodextrin or a combination thereof. In one embodiment, the binding composition consists essentially of maltitol, allulose, resistant starch or a combination thereof.

In one embodiment, the binding composition is substantially sugar-free. In one embodiment, the binding composition is substantially free of any sugar having a glycemic index of more than 50. In one embodiment, the binding composition is substantially free of sugar alcohols.

In one embodiment, the binding composition has a glycemic index of more than 70. In one embodiment, the binding composition has a glycemic index of 220, 200, 170, 160, 120, 100, 80, or 70. In one embodiment, the binding composition has a glycemic index of less than 50, 30, 20, 15, 10, 8 or 5. In one embodiment, the binding composition has a glycemic index of 0. In one embodiment, the chewable composition may have zero calorie.

In one embodiment, the chewable composition has a glycemic index of more than 70. In one embodiment, the chewable composition has a glycemic index of 220, 200, 170, 160, 120, 100, 80, or 70. In one embodiment, the chewable composition has a glycemic index of less than 50, 30, 20, 15, 10, 8, or 5. In one embodiment, the chewable composition, having a glycemic index of about 0.

In one embodiment, the chewable composition comprises from about 50% to 85% by weight of the binding composition. In one embodiment, the chewable composition comprises from about 60% to 80%, 65% to 75%, 65% to 60%, 67% to 71% or 68% to 69% w/w of the binding composition. In one embodiment, the chewable composition comprises from about 50%, 67%, 68%, 70%, 75%, 80% w/w of the binding composition.

Through extensive research, the applicant discovered that the semi-solid chewable gel compositions comprising essentially sugar alcohol or low glycemic sugars as binding agents are prone to crystallization leading to insatiability of the formulation. The applicant further discovered that certain concentration of polymer stabilizers may be used to promote the thermodynamic stability of the formulation and reducing the crystallization.

Through significant and extensive experimentation, Applicant discovered that a stabilizing polymer significantly extends the stability of a low-sugar or sugar-free chewable composition formulations for about 1.5, 2, 3, 4, 5, 7, 8, 9, or over 10 times when compared to same formulations without the polymer stabilizer. For example, the stability may be extended from about 2 weeks to about 8 months, about 3 months to about 9 months, about 6 months to about 12 months, about 5 months to about 14 months, about 9 months to over 24 months, about 10 months to over 36 months.

The polymer stabilizer may include a polysaccharide, a polyvinyl alcohol, a polyalcohol, a vinyl alcohol, a peptide, a cationic polymer, a polyphenol, or a combination thereof.

In one embodiment, the polymer stabilizer may be a polymer of monosaccharide monomers selected from glucose, fructose, mannose, galactose, arabinose, rhamnose, xylose, galacturonate, glucuronate, N-acetylgalactosamine, N-acetylglucosamine, or a combination thereof, and wherein the polymer comprises from about 5 to about 500 monosaccharide monomers. In one embodiment, the polysaccharide comprises from about 5 to about 50 monomers.

In one embodiment, the monosaccharide monomer comprises glucose monomer or mannose monomer linked through glyosidic bonds substantially free of alpha-1,4-glycosidic bond. In one embodiment, the glycosidic bond comprises 1,2-alpha glycosidic bond, 1,3-alpha glycosidic bond, 1,2-beta glycosidic bond, 1,3-beta glycosidic bond, or a combination thereof. In one embodiment, the polysaccharide comprises alpha-mannose monomers, beta-mannose monomers, beta-glucose monomers, or a combination thereof.

In one embodiment, the polymer stabilizer may be a polysaccharide. The polysaccharide may be cationic, anionic, or nonionic. It could be homo-polysaccharide or hetero-polysaccharide.

In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccharide, fructo-oligosaccharide, galactomannan oligomers, oligomers of ribose, xylose, arabinose, rhamnose, or a combination thereof.

In one embodiment, the polymer stabilizer comprises soluble fiber from tapioca, soluble corn fiber, soluble fiber from chicory root, soluble fiber from dandelion, maltodextrin, resistant maltodextrin, 6-20 B3-1,4-linked glucopyranose units, 6-20 β-1,3-linked glucopyranose units, 6-20 β-1,2-linked glucopyranose units, 6-20 a-1,3-linked glucopyranose units, 6-20 a-1,2-linked glucopyranose units, or combination thereof. In one embodiment, the polymer stabilizer comprises maltodextrin.

In one embodiment, the vinyl alcohol comprises a hydroxy methyl acrylate.

In one embodiment, the peptide comprises a collagen, a cationic peptide, or a combination thereof.

In one embodiment, the chewable composition includes from about 1% to 10%, 1% to 5%, 2% to 6%, 0.5% to 15% w/w of the polymer stabilizer. In one embodiment, the chewable composition comprises at least 2% w/w of the polymer stabilizer. In one embodiment, the chewable composition comprises from about 3% to 10%, 5% to 8%, 6% to 7% w/w of the polymer stabilizer.

In one embodiment, the binding composition consists essentially of isomalt, allulose, or a combination thereof with resistant maltodextrin as the stabilizer. In one embodiment, the binding composition consists essentially of isomalt, maltitol, or a combination thereof with polydextrose as the stabilizer.

In one embodiment, the weight ratio of the binding composition and the polymer stabilizer is from about 3:1 to about 20:1, or any ratio in between, including 5:1, 6:1, 7:1, 8:1, 10:1, 12:1, 15:1, 18:1, or 19:1. In one embodiment, the weight ratio of the binding composition and the polymer stabilizer from about 10:1 to 15. In one embodiment, the weight ratio of the binding composition and the polymer stabilizer may be from about 5:1 to 20:1, 6:1 to 15:1, 5:1 to 10:1, 10:1 to 12:1, 12:1 to 15:1, 10:1 to 20:1, or from 8:1 to 18:1.

In one embodiment, the gelling composition comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

In one embodiment, the gelling composition comprises pectin. In one embodiment, the pectin has a methoxyl content (i.e., esterification degree or DE) not less than about 15%, 20%, 40%, 50% or 65%. In one embodiment, the methoxyl content is from about 15% to 40%, 15% to 25%, 16% to 24%, 30% to 70%, 50% to 65%, 55% to 65%, 59% to 63%, or 60% to 80%.

In one embodiment, the pectin has an amide content not less than about 15%, 20%, 30%, or 40%. In one embodiment, the amid content is from about 12% to 40%, 15% to 35%, 15% to 25%, 20% to 25%, 25% to 40%.

In one embodiment, the total of the methoxyl content and the amide content is from about 36% to 70%.

In one embodiment, the methoxyl content is more than about 25% and the amide content is not less than about 20. In one embodiment, the methoxyl content is from about 16% to 24% and the amide content is from about 20% to 25%. In one embodiment, the methoxyl content is from about 56% to 66% and the amide content is from about 0.1% to 0.5%.

In one embodiment, the gelling composition comprises gelatin. In one embodiment, the gelling composition comprises pectin, gelatin, collagen, or a combination thereof. In one embodiment, the gelling composition comprises pectin and collagen in a ratio from about 1:1 to about 1:3. In one embodiment, the gelling composition comprises pectin and collagen in a ratio about 1:2.

In one embodiment, the chewable gel composition includes from about 0.5% to 10%, 1.5% to 2.5%, 0.5% to 1.5% w/w of the gelling composition. In one embodiment, the chewable gel composition includes from about 0.5% to 1.5% w/w of the gelling composition, wherein the gelling composition comprises carrageenan. In one embodiment, the semi-solid chewable gel composition includes from about 5% to about 10% by weight of the gelling composition, wherein the gelling composition comprises gelatin.

In one embodiment, the semi-solid chewable gel composition may further comprise an active pharmaceutical ingredient (API), a complexing composition, an herbal composition, an antioxidant composition, a vitamin composition, a mineral composition, an amino acid composition, a probiotics composition, or a prebiotics composition.

In one embodiment, the API composition comprises a cannabinoid, an antibiotics, an anti-histamine, an anti-inflammatory agent, an fever reducer, a painkiller, a psychoactive agent, an aldosterone receptor antagonist, an angiotensin converting enzyme inhibitor, an angiotensin receptor blocker, a neprilysin inhibitor, an antiadrenergic agent, an antianginal agent, an antiarrhythmic agent, an anticholinergic chronotropic agent, an antihypertension agent, an ACE inhibitor, an angiotensin II inhibitor, an antiadrenergic agent, a beta blocker, a diuretic agent, a beta-adrenergic blocker, a calcium channel blocker, a catecholamine, an inotropic agent, a vasodilator, a renin inhibitor, a sclerosing agent, a vasopressin antagonist, a vasopressor, an anti-cholesterol agent, a statin, an agent for dyslipidemias, an antiplatelet, an anticoagulant, an antianginal agent, a corticosteroid, a beta agonist, a proton pump inhibitor, a laxative, a respiratory agent, an anti-diarrhea agent, an anti-ulcerative colitis agent, an anti-nausea agent, a renal medication, an anti-epilepsy agent, an analgesics, a muscle relaxants, an antipsychotics, a benzodiazepine, a selective serotonin reuptake inhibitor (SSRI), a diabetic agent, an antidepressant, an anti-anxiety agent, an anti-tumor agent, a stimulant, a contraceptive, a corticosteroid, an alpha blocker, a 5-alpha reductase inhibitor, a osteoporosis drug, an immune-suppressant, a PED5 inhibitor, an overactive bladder drug, an anti-gout drug, an anti-glaucoma drug, an expectorant, a cough suppressant, a sleep aid, an antifungal agent, an anti-viral agent, or a combination thereof.

In one embodiment, the API composition comprises cannabidiol (CBD) to provide a chewable composition having about 20 mg-1500 mg per dose. In one embodiment, the chewable composition has dosage of CBD at 40 mg, 80 mg, 100 mg, 200 mg, 400 mg, 500 mg, 600 mg, 800 mg, 1000 mg, 1500 mg or 2000 mg.

In one embodiment, the API composition comprises caffeine, melatonin, glutathione, nicotine, cannabidiol, acetaminophen, aspirin, salicylic acid, ibuprofen, naproxen, diphenhydramine, epinephrine, scopolamine, metformin, cetirizine, loratadine, chlorpheniramine, brompheniramine, alimemazine, cyprohetadine, doxylamine, hydroxyzine, promethazine, guaifenesin, codeine phosphate, dextromethorphan hydrobromide, acamprosate, baclofen, buprenorphine, naloxone, clonidine, disulfiram, methadone, naltrexone, ondansetron, bupropion, cytosine, varenicline, citalopram, clomipramine, dexopin, escitalopram, fluoxetine, fluvoxamine, imipramine, mirtazapine, paroxetine, sertraline, trazodone, amitriptyline, doxepin, mianserin, mirtazapine, trazodone, trimipramine, tadalafil, sildenafil, vardenafil, avanafil, sumatriptan, amoxilin, clindamycin, erythromycin, rapamycin, febuxostat, fexofenadine, fomepizole, orlistat, or a combination thereof.

In one embodiment, the API composition comprises caffeine to provide a chewable composition having about 50 mg-400 mg per dose. In one embodiment, the chewable composition has caffeine dosage of 100 mg, 150 mg, 200 mg, 250 mg, 300 mg, or 400 mg per dose.

In one embodiment, the herbal composition comprises, per dose, at least 50 mg, 100 mg, 150 mg, 200 mg, 300 mg of glutathione and at least 10 mg, 10 mg, 25 mg, 30 mg, or 50 mg milk thistle extract or powder.

In one embodiment, the herbal composition comprises an adaptogen. In one embodiment, the herbal composition comprises butterbur, quercetin, Stinging nettles (Urtica dioica), bromelain, phleum pratense, tinospora cordifolia, elderflower, elderberry, sorrel, cowslip, Verbena, gentian root, echinacea, grape seed, pycnogenol, pine bark extract, EPA, honey, cat's claw, albizzia (Albizzia lebbeck), baical skullcup (Scutellaria baicalensis), goldenseal, spirulina, bitter orange (citrus aurantium), lemon, eucalyptus, frankincense, Angelica sinensis, eyebright (Euphrasia officinalis), Gingko, milk thistle (Silybum marianum), red clover (Trifolium pratense), Yarrow (Achillea millefolium), rosemary, shiso, sage, peppermint, licorice, Artemisia argyi, Stephania tetrandra, coix seed, Citrus trifoliata, Citrus aurantium, Angelica dahurica, ashwagandha, maca, yinyan huo (horny goat weed), variety of ginsengs such as America ginger, panax ginseng, Siberia ginseng, extract, powder, isolate or distillate thereof.

In one embodiment, the herbal composition comprises, per dose, at least 100 mg, 200 mg, 500 mg of gingseng or ashwagandha.

In one embodiment, the herbal composition comprises antioxidant herbs, their extract, powder, isolate or distillate thereof. Examples include without limitation clove, peppermint, allspice, cinnamon, oregano, thyme, sage, rosemary, saffron, estragon, Ginkgo biloba, licorice, schizandra, quercitin, plantain, opium poppy, cocoa, coffee.

In one embodiment, the herbal composition comprise Ginkgo biloba and cocoa and the API composition comprises caffeine to provide a chewable composition comprising, per dose, at least 50, 100 mg, 150 mg, 200 mg of caffeine, at least 200 mg, 300 mg, 500 mg, 800 mg of cocoa and at least 10 mg 20 mg, 40 mg, 100 mg of Ginkgo biloba.

In one embodiment, the herbal composition comprises immunity support herbs, their extract, powder, isolate or distillate thereof. Examples include without limitation elderberry, goji berry, holy basil, echinacea, ashwagandha, mulethi, amla, various medicinal mushrooms such as reishi, lion's mane, etc.

In one embodiment, the herbal composition comprises, per dose, at least 200 mg, 300 mg, 400 mg, 500 mg, 600 mg, or 800 mg of elderberry extract or powder and at least 20 mg, 30 mg, 35 mg, 50 mg, 80 mg, 100 mg of gojiberry extract or powder.

In one embodiment, the herbal composition comprises anti-aging herbs, their extract, powder, isolate or distillate thereof. Examples include without limitation astragalus, angelica.

In one embodiment, the herbal composition comprises, per dose, at least 200 mg, 300 mg, 400 mg, 500 mg, 600 mg, 800 mg, 900 mg, or 1000 mg of astragalus extract or powder. In one embodiment, the herbal composition comprises, per dose, at least 20 mg, 40 mg, 100 mg, 200 mg, 250 mg, or 500 mg of anglica extract or powder.

In one embodiment, the herbal composition comprises anti-inflammatory herbs, their extract, powder, isolate or distillate thereof. Examples include without limitation ginger, turmeric, curcumin, boswellia serrate resin (Frankincense), white willow bark, green tea, pycnogenol (maritime pine bark), resveratrol, uncaria tomentosa (cat's claw), any ginseng species such as American ginseng, red panax ginseng, or Siberia ginseng, ashwagandha, Schisandra, Ginkgo Biloba, Cayenne, capsaicin (chilli pepper), black pepper, rosemary, Devils claw (Harpagophytum procumbens), Feverfew, Arnica montana, Hypericum (St John's Wort) chamomilla, bromelain, clove, cinnamon, thyme, Astragalus.

In one embodiment, the herbal composition comprises, per dose, at least 100 mg, 200 mg, 400 mg, 500 mg, 600 mg, 800 mg or 1000 mg of curcumin or turmeric and at least 10 mg, 20 mg, 25 mg, 50 mg, 100 mg of ginger powder or extract.

In one embodiment, the herbal composition comprises male stamina or testosterone enhancing herbs, their extract, powder, isolate or distillate thereof. Examples include without limitation ginger, prickly ash bark, turmeric, motherwort, eleuthero, any ginseng variety including without limitation American ginseng, panex ginseng, or red panax ginseng, ashwagandha, Schisandra, wild oats, passion flower, Valerian, Chinese yam (Dioscorea sp), Eucommia (Eucomnia ulmoides), Ginkgo Biloba, Deer antlers (Cervi pantotrichum), Seahorse (Hippocampus kelloggii), Tribulus terrestris, Tongkat Ali (Eurycoma longiflora), Gambir, Muira puama (Ptychopetalum olacoides), Yohimbe (Pausinystalia yohimbe), Epimedium (Horny Goat Weed), Maca (Lepedium meyenii W), Oatstraw, Catuaba (Erythroxylum catuaba), Nettle leaf (Urtica dioica), Dadder seed (Tusizi), Cordyceps (Dongchongxiacao), Cayenne, Astragalus, Sarsparilla (Smilax officinalis), Licorice root, Pumpkin seed, Yohimbe, Eurycoma longifolia, beet.

In one embodiment, the herbal composition comprises, per dose, at least 100 mg, 200 mg, 400 mg, 600 mg, or 800 mg of Epimedium extract.

In one embodiment, the herbal composition may include mood enhancing or energizing herbs, their extract, powder, isolate or distillate thereof. Example herbs include without limitation rhodiola, schisandra, shilajit, eleuthero, cordyceps, lion's mane, gynostemma, burdock, chircory, dandelion roots, cacao, shatavari, condonopsis pilosula, atractylodes macrocephala Koidz, he shou wu, Ceylon cinnamon, gac (Momordica cochinchinensis), Astragalus, Ginseng, licorice, and red jujube.

In one embodiment, the herbal composition may include sleep-promoting or calming herbs, their extract, powder, isolate or distillate thereof. Example herbs include ziziphus jujuba seeds, valerian root, passionflower, ashwagandha, gingko biloba, chamomile, kava, holy basil, St. John's Wort, wild lettuce, hops, mint, lemon balm, California poppy, magnolia bark, mimosa, nutmeg, cinnamon, marjoram, mimosa (Albizia julibrissi), fig, shatavari, rehmannia, ashwagandha, and reishi. In one embodiment, the sleep-promoting herb may be magnolia (Magnolia officinalis, Magnoliaceae), Semen zizyphi spinosae (Zizyphus jujube Mill. var. spinosa Rhamnaceae), sinomenine (Sinomenium acutum Menispermaceae), decursinol (Angelica gigas, Unbelliferae), rosemary (Perilla frutescens, Lamiaceae), Euphoria longan (Spindaceae), Ginseng (Panax ginseng, Araliacae), EGCG (Epigallocatechin-3-O-gallate, Camellia sinensis), Chrysanthemum morifolium (Asteraceae), and Apigenin (Circiumjaponicum, Asteraceae).

In one embodiment, the chewable composition comprises, per dose, at least 100 mg, 200 mg, 250 mg, 350 mg, 500 mg ziziphus jujuba seed extract or powder, at least 250 mg, 300 mg, 500 mg, 600 mg, 800 mg valerian root extract or powder. In one embodiment, the chewable composition may further comprise, per dose, 1 mg, 2 mg, 3 mg, 5 mg, or 10 mg of melatonin.

In one embodiment, the herbal composition may have the effect of promoting gut or digestive track health. In one embodiment, the herbal composition may include apple cider vinegar. The apple cider vinegar may include the mother. In one embodiment, the herbal composition may include other gut or digestive track health promoting herbs such as ginger, turmeric, cinnamon, bay leaves, slippery elm, clove, oregano, cardamom, cumin, funnel, peppermint, extract, powder, isolate or distillate thereof.

In one embodiment, the herbal composition comprises, per dose, at least 800 mg, 1000 mg, 1250 mg, 1500 mg, 1750 mg, or 2000 mg of apple cider vinegar.

In one embodiment, the herbal composition may have the effect of promoting weight loss. Example herbs include black pepper, fenugreek, Caralluma Fimbriata, Gymnema sylvestre, green coffee bean, green tea, dandelion, psyllium, Garcinia cambogia, extract, powder, isolate or distillate thereof.

In one embodiment, the complexing composition may be configured to complex with an API or herbal composition therefore masking or modulating the flavor profile or reducing bitterness of the semi-solid chewable gel composition.

The complexing composition is capable of interacting with API and forming an API-complex. The complexing composition is capable of complexing with API through coordinating, chelating, complexing, hydrogen-bonding, dipole-dipole interaction, van-der waals interaction, or a combination thereof. In one embodiment, the API complex is capable of masking, lessening or reducing the antihistamine's taste, increasing antihistamine's solubility or stability in aqueous matrix, or a combination thereof. In one embodiment, the API complex is capable of masking and reducing the bitterness, astringent or metallic taste of the antihistamine. In one embodiment, the API complex is capable of increasing API's solubility in aqueous matrix therefore facilitating the incorporation of API into the aqueous gummy matrix.

In one embodiment, the complexing composition may be a cyclic glucose molecule (alpha-, beta-, gamma-cyclodextrin), cluster dextrin, maltodextrin, resistant starch, an oligosaccharide (such as inulin or soluble or non-soluble dietary fiber), a polysaccharide (such as a herbal polysaccharide), nucleic acid (DNA or RNA), an nucleotide molecule, an amino acid or its derivative thereof, a peptide, or an amid. In one embodiment, the complexing composition comprises cyclodextrin, a nucleotide, resistant starch, or a combination thereof.

In one embodiment, the complexing composition comprises protein, peptide, amide or polyamide, cluster dextrin, cyclodextrin, polydextrose, resistant starch, polyethylene glycol, polyunsaturated hydrocarbons, polyunsaturated fatty acids, mica, talc, zeolite, cellulose, plant particles, calcium carbonate, diatomaceous earth, chitosan, or a combination thereof. In one embodiment, the complexing composition comprises cyclodextrin, a nucleotide, resistant starch, an amide, a peptide, or a combination thereof.

In one embodiment, the complexing composition comprises an amide. Example amide includes without limitation N-acetyl glucosamine, n-acetyl galactosamine, 2-deoxy-2-aminoglucose N-acetyl, sialic acid N-acetyl, iminosugar N-acetyl, daunosamine N-acetyl, 2-deoxy-2aminogalactose N-acetyl, chitin, pectin, and amino acids.

Plant particles may be derived from various parts of a plant such as flower, fruit, seed, grain, nut, nutshell, root, leaves, or stems. In one embodiment, the plant particles comprise berry powder, nutshell powder, rice bran powder including without limitation strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, cranberry powder or blueberry powder. DNA rich plant powder may be preferred such as strawberry, which is an octoploid. In one embodiment, the complexing composition comprises strawberry DNA.

In one embodiment, the complexing composition comprises cluster dextrin or cyclodextrin. In one embodiment, the complexing composition comprises cyclodextrin. In one embodiment, the cyclodextrin comprises alpha-dextrin, beta-cyclodextrin, gamma-cyclodextrin, or a combination thereof. In one embodiment, the cyclodextrin comprises essentially gamma-cyclodextrin.

In one embodiment, the composition comprises API and cyclodextrin at a molar ratio of from about 1:1 to about 1:100, from about 1:1 to about 1:20, or any ration in between. In one embodiment, the molar ratio of API and cyclodextrin is about 1:2, 1:5, 1:8, or 1:10.

In one embodiment, the antioxidant composition comprises glutathione, Vitamin E, Vitamin C, beta-carotene, gallic acid, selenium, selenium yeast, phenolics, anthocyanins, flavonoids, polyphenols, whey, bioflavonoids, theobromine, anthracenes, carotenoids, lutein, zeaxanthin, ginko biloba, berry extract, resveratrol, saffron, Sangre de grado (dragon's blood), cocoa, or derivatives thereof In one embodiment, the vitamin composition comprises vitamin A, B, C, D, E, K or a combination thereof. In one embodiment, the vitamin composition comprises Vitamin B9 (or folic acid), Vitamin D, Vitamin B3 (Niacin or Niacinamide), Vitamin C, or a combination thereof.

In one embodiment, the mineral composition comprises salts of calcium, iron, zinc, magnesium, sodium, chloride, potassium, copper, molybdenum, manganese, phosphorus, iodine, nickel, or selenium, or a combination thereof. In one embodiment, the mineral composition comprises zinc citrate, zinc gluconate, zinc sulfate, zinc acetate, boron citrate, or a combination thereof.

The amino acid may be natural or non-natural occurring. In one embodiment, the amino acid composition comprises histidine, a branched chain amino acid (leucine, iso-leucine, valine), L-5 hydroxytryptophan (5-HTP), an essential amino acid such as histidine, lysine, methionine, phenylalanine, threonine, and tryptophan, L-theanine, beta-alanine, or its derivative thereof. In one embodiment, the amino acid composition may include a sleep promoting or calming amino acid including, for example, glycine, tryptophan, L-theanine, or a derivative thereof.

In one embodiment, the prebiotic composition comprises gum arabic, chicory root powder or extract, wheat bran powder or extract, acacia gum, guar gum, Artichoke fiber, oat fiber, soluble corn fiber, inulin, resistant maltodextrin, resistant starch, or a combination thereof.

In one embodiment, the probiotic composition comprises gum arabic, chicory root powder or extract, wheat bran powder or extract, acacia gum, guar gum, Artichoke fiber, oat fiber, soluble corn fiber, inulin, resistant maltodextrin, resistant starch, or a combination thereof. In one embodiment, the probiotic composition comprises Lactobacillus acidophilus, Lactobacillus rhamnosus GG, Saccharomyces boulardii, Bifidobacterium bifidum, Bacillus coagulans, or a combination thereof.

In one embodiment, the semi-solid chewable gel composition may further comprise an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the sweetener comprises xylitol, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl)propyl]-L-.alpha.-aspartyl]-L-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

In one embodiment, the semi-solid chewable gel composition is substantially free of artificial sweeteners or sugar substitutes.

In one embodiment, the chewable composition may further include a coating composition. In one embodiment, the coating composition comprises isomalt, allulose, tagatose, xylitol, erythritol, sorbitol, mannitol, or a combination thereof. In one embodiment, the coating composition may have a particle size from about 0.6 mm to about 0.75 mm. In one embodiment, the coating composition may have a particle size of about 400 microns.

In one embodiment, the chewable composition may include the binding composition comprising maltitol and at least one of allulose, xylitol, erythritol, maltitol, sorbitol, and mannitol, and the coating composition including maltitol.

The pH of the chewable composition may be from about 3 to 5, 2 to 6, 2 to 4, 3 to 4, 2 to 5, or any number in between. In one embodiment, the pH of the composition is less than 5. In one embodiment, the pH of the composition is more than 7. In one embodiment, the pH of the composition is about 9.

In one embodiment, the gelling composition comprises pectin. In one embodiment, the pH of the composition is less than 3, 3.5, or 4. In one embodiment, the pH of the composition is more than 3 or 3.5. In one embodiment, the pH of the composition is more than 4 or 4.5. In one embodiment, the pH of the composition is about 7.

In one embodiment, the gelling composition comprises gelatin. In one embodiment, the pH of the composition is more than 6.

In another aspect, the application provides methods of making the chewable composition. In one embodiment, the method includes the steps of dividing the binding composition into a first binding portion and a second binding portion, combining a first mixture and water and heating to at a first elevated temperature to provide a first solution, wherein the first mixture comprises the first binding portion and optionally the polymer stabilizer, combining the second mixture and water at a second elevated temperature to provide a second solution, wherein the second mixture comprises the gelling composition with the second binding composition that is equal to, twice, three time, or four times the mass of the gelling composition, and the complexing composition, mixing the second solution into the first solution at a third elevated temperature to provide a third mixture, wherein the third mixture has a Brix number from about 80 to about 85 or from about 78 to about 86, adjusting pH of the third solution with a buffer salt to from about 3 to about 7.

In one embodiment, the method may further include the step of adding coloring agent, flavoring agent, or a combination thereof into the third mixture to provide a molding mix having a Brix from about 78 to about 86.

The API composition and the complexing composition may be added to the second mixture, the third mixture, or the molding mixture. In one embodiment, the API composition and the complexing composition may be added to the third mixture together with the coloring agent or flavoring agent.

In one embodiment, the method may further include the step of adding the molding mix to a preformed shaped cavity. In one embodiment, the method may further include the step of cooling the molding mix (or mixture) in the preformed shaped cavity until the molding mixture forms into the chewable composition piece.

The first, second and third elevated temperature may be independently from about 175° F. to about 275° F., from 175° F. to about 200° F., from 170° F. to about 210° F., or any temperature in between.

In one embodiment, the method comprises the following steps. In a first container, a gelling composition is mixed with a portion of a binding composition. Optionally, a buffering salt may be added. The components are mixed until homogeneous to provide a first mixture (Mix 1). In one embodiment, the gelling composition comprises pectin. In one embodiment, the portion of the binding composition comprises sorbitol and isomalt, or allulose and maltitol. In one embodiment, the buffering salt may include sodium citrate, potassium citrate, or a combination thereof In a second container, the remaining portion of the binding compositions added. In one embodiment, the remaining portion of the binding composition comprises sorbitol, isomalt, and mannitol. The components are mixed until homogeneous to provide a second mixture (Mix 2).

In a third container, a food acid is dissolved in an aqueous solution with additives such as coloring agent and flavoring agent. In one embodiment, the food acid comprises citric acid, malic acid, acetic acid, or a combination thereof. In one embodiment, the aqueous solution comprises water, ethanol, glycerol, or any combination thereof. All components are mixed and warmed until a homogenous solution is achieved to provide a third mixture (Mix 3). In one embodiment, the components are warmed to 175° F.

To a first reaction container, the API composition and the complexing composition may be added. In one embodiment, the API composition comprises acetaminophen or a derivative thereof. In one embodiment, the complexing composition comprises N-acetyl glucosamine and beta-cyclodextrin. Water is then added to the first reaction container. In one embodiment, the water may be heated first before adding to the first reaction container. In one embodiment, the water is heated to at least 200° F.

Then the Mix 1 is added to the first reaction container with stirring to provide a first solution. The mixture is stirred until the gelling composition fully swells and disperses. In one embodiment, the first solution may be brought to a light boil.

To a second reaction container, Mix 2 is added, followed with addition of water. The components are mixed to provide a second solution. In one embodiment, the second solution may be brought to a boil.

Then, the first solution is combined with the second solution with mixing. The mixture is heated to a Brix number of at least 82 Brix. Then, Mix 3 is added dropwise with stirring to provide a molding mix having a Brix number of at least 82 Bix.

The molding mix was then added to a mold to provide individual gummy pieces. The mold may be a silicon mold, a starch mold, or a sugar alcohol mold. In one embodiment, the sugar alcohol mold is made by compacting a sugar alcohol composition powder or particles in a container to create a compacted a sugar alcohol mass, and stamping the compacted mass with a desirable shape to create mold cavities in the compacted mass. In one embodiment, the sugar alcohol composition comprises maltitol, isolmalt, or a combination thereof.

The molding mix may be injected or deposited into the mold cavities to form gummy pieces. The gummy piece may be any shape and size. The formed chewable composition may be in square shape, gumdrop shape, hexagon shape, partial ball shape, animal shape, cartoon shape, or any desirable shape. The shape may be hexagon, square, half ball, gumdrop, heart, bear, or any other shapes.

The formed composition may be from about 1 g to about 10 g, from about 2 g to about 7 g, from about 3 g to about 5 g, or any number in between. In one embodiment, the gummy piece may have a weight from about 3 g, 4 g, 5 g, 6 g, 7 g, 7.5 g, to about 8 g.

The gummy piece may be further coated with a coating composition. The coating composition may prevent gummy piece to stick with each other. The coating composition may include isomalt, maltitol, or other low glycemic sugar or sugar alcohol. In one embodiment, the coating composition comprises isomalt. In one embodiment, the coating composition comprises maltitol.

Through extensive experimentation, the Applicant discovered that the water solubility property of the coating composition is critical in maintaining gummy formulation stability. In one embodiment, the coating composition may have a water solubility of at least 2000 g/L, 1750 g/L, 1500 g/L, 1000 g/L, 500 g/L of water at room temperature. In one embodiment, the water solubility of the coating composition is at least 1500 g/L. In one embodiment, the coating composition comprises isomalt, allulose, maltitol, maltodextrin, inulin, starch, bran, xylitol, sorbitol, tagatose, erythritol, or a combination thereof.

The gummy shape may be geometric or in animal, or any object shape. Example shape includes hexagon, square, half ball, gumdrop, heart, bear, cube, square, half ball, bar, or round disk.

The mold may be a silicon mold, a starch mold, or a sugar alcohol mold. In one embodiment, the sugar alcohol mold is made by compacting sugar alcohol powder or particles in a container to create a compacted a sugar alcohol mass and stamping the compacted sugar alcohol mass with a desirable shape to create mold cavities in the compacted sugar alcohol mass. The molding mix may be injected or deposited into the mold cavities to form gummy pieces. The sugar alcohol may be maltitol, isomalt or a combination thereof.

In another aspect, the application provides methods of treating a disease or modulating a physiological condition in a subject using the semi-solid chewable gel composition. In one embodiment, the physiological condition may allergy, insomnia, inflammation, diabetes, obesity, cardiovascular, or a combination thereof. In one embodiment, the method includes the step of administrating an effective amount of the semi-solid chewable gel composition to the subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the ring structure of the cyclodextrin complexing with the cetirizine molecule;

FIG. 2 shows the chemical structure of Famciclovir, in which the fused ring system is a derivative of the nucleic base guanosine; and

FIG. 3 shows the interaction of a nucleotide with an API for flavor modulation.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, and processes related to semi-solid chewable composition.

### Gelling Composition

In one embodiment, the gelling composition comprises pectin, gelatin, or a combination thereof. In one embodiment, gelatin may be combined with other hydrocolloids-pectin, agar, starch, gum Arabic—to create desired textures. In one embodiment, gelatin may be combined with gum arabic as the gelling composition.

In one embodiment, the gelling composition comprises starch such as amylose starch or modified starch. There are a variety of starch modification techniques, such as contacting starch with acid, sodium or potassium hydroxide, or oxidizing the starch.

In one embodiment, the gelling composition comprises agar. Agar may be combined with locust bean gum as a gelling composition. Locust bean gum helps to prevent weeping of agar gels. The two polysaccharides from agar and locust bean gum synergistically interact with each other to form a strong gel that does not weep.

In one embodiment, the gelling composition comprises carageenans. Carrageenans or carrageenins are linear sulfated polysaccharides. Kappa-carrageenan has one sulphate group per disaccharide and forms strong, rigid gels in the presence of potassium ions. In one embodiment, locust bean gum may be used with kappa-carrageenan to prevent weeping. Gels formed from kappa-carrageenan and potassium ions are thermally reversible.

In one embodiment, the gelling composition comprises alginic acid or alginate. Alginate may form strong hydrogels when crosslinked with calcium ions.

### Binding Composition

Binding composition binds the semi-solid chewable gel (gummy) together through interaction with the gelling composition. The interaction may be through hydrogen bonding or through covalent bonding. In one embodiment, the binding composition comprises sugars, sugar derivatives, sugar alcohols, or a combination thereof. The binding composition may keep texture of the product soft by acting as a humectant.

Conventional gummy formulation typically contains 60-85% w/w sugars, leading to high glycemic index (GI) formulation that is not safe for diabetic population. Moreover, conventional sugars such as glucose, sucrose, and fructose are cariogenic and high in both glycemic index and calorie. Thus, for many situations, it would be advantageous to have a sugar free gummy or a low GI gummy formulation as disclosed thereof. In one embodiment, the disclosed composition does not cause tooth decay.

In one embodiment, the binding composition comprises a mono- or di-saccharide (i.e., sugar) having a glycemic index of less than 35, 30, 25, 20, 15, or 10, a sugar alcohol, or a combination thereof. In one embodiment, the semi-solid chewable composition is substantially free of sugar having a glycemic index of more than 35. In one embodiment, the semi-solid chewable composition has a glycemic index of not more than 8, 10, 15 or 20.

In one embodiment, the semi-solid chewable composition is substantially free of sucrose, fructose, glucose, sugar alcohol, sugar substitute, or non-sugar sweetner. Example sugar substitutes include, without limitation, sucralose, stevia extract or derivatives, monk fruit extract, licorice extract or derivative, tamarind extract or derivative, or their derivative thereof.

In one embodiment, the binding composition comprises a low GI sugar having a glycemic index (GI) of not more than 18, 20, 30 or 35. In one embodiment, the low GI sugar comprises allulose, sorbose, tagatose, trehalose, isomaltulose, raffinose, or a combination thereof. In one embodiment, the binding composition comprising tagatose, allulose (also known as allulose), sorbose, isomaltulose (also know as isomaltulose), trehalose (also known as mycose), mannose, maltose, ribose, xylose, tetroses, pentoses, hexoses, heptoses, their acid forms or a combination thereof. In one embodiment, the binding composition consists essentially of allulose, isomaltulose, and a third low GI sugar selected from a group consisting of trehalose, sorbose, tagatose, or a combination thereof. In one embodiment, the binding composition further comprises N-acetyl glucosamine.

Allulose is identical to sucrose in sweetness but has nearly zero calories and does not promote tooth decay. Sorbose is equivalent to sucrose in sweetness. Tagatose is nearly as sweet as sucrose yet only has 38% of the caloric value of sucrose and is much more tooth friendly than sucrose. Trehalose is a non-reducing sugar with reported antioxidant effects and significant neurological benefits. Isomaltulose is slowly metabolized by the body leading to a low insulin index and also has a lower instances of tooth decay.

Through extensive experimentation, processes are developed to used allulose, sorbose, tagatose, isomaltulose, trehalose in the gummy formulation allowing these sugars behave like conventional sugars such as sucrose and fructose but without the caloric significance of the convention sugars.

In one embodiment, the binding composition comprises allulose, trehalose and isomaltulose. In one embodiment, the binding composition comprises more than 20% isomaltulose. In one embodiment, the binding composition comprises from 15% to 35% isomaltulose. In one embodiment, the binding composition comprises not more than 75% of allulose. In one embodiment, the binding composition comprises from 45% to 60% allulose. In one embodiment, the binding composition comprises not more than 45% of trehalose.

In one embodiment, the binding composition comprises allulose and tagatose. In one embodiment, the binding composition comprises not more than 50% tagatose. In one embodiment, the binding composition comprises from 30% to 45% tagatose. In one embodiment, the binding composition comprises not more than 70% of allulose.

In one embodiment, the binding composition comprises isomaltulose and tagatose. In one embodiment, the binding composition comprises from 30% to 60% tagatose. In one embodiment, the binding composition comprises not more than 70% of isomaltulose.

Sugar alcohols are sweet, non-cariogenic, not digestible and provide fewer calories than many sugars. In some embodiments, sugar alcohols may mask other flavors. For example, mannitol may be used to mask bitterness. Mannitol masks bitterness by a mechanism that involves the endothermic nature of mannitol dissolving into water.

In one embodiment, the binding composition comprises essentially of sugar alcohols. Example sugar alcohols include sorbitol, mannitol, erythritol, xylitol, isomalt, maltitol, lactitol, and hydrogenated starch hydrolysates. In one embodiment, the binding composition comprises mannitol, maltitol, or isomalt. In one embodiment, the binding composition comprises mannitol, sorbitol, isomalt, or a combination thereof. In one embodiment, the binding composition consists essentially of mannitol, maltitol, sorbitol, or a combination thereof. In one embodiment, the binding composition consists essentially of mannitol, maltitol, xylitol, or a combination thereof. In one embodiment, the binding composition consists essentially of mannitol, xylitol, isomalt, or a combination thereof. In one embodiment, the binding composition consists essentially of mannitol, xylitol, sorbitol or a combination thereof. In one embodiment, the binding composition consists essentially of mannitol, sorbitol, isomalt, resistant starch or a combination thereof. In one embodiment, the binding composition consists essentially of mannitol, maltitol, sorbitol, maltodextrin or a combination thereof. In one embodiment, the binding composition consists essentially of mannitol, sorbitol, or a combination thereof. In one embodiment, the binding composition consists essentially of mannitol, sorbitol, erythritol or a combination thereof.

Mannitol has 50-70% of the relative sweetness of sugar. Mannitol lingers in the intestines for a long time and therefore may cause gastric distress.

Erythritol ((2R,3S)-Butan-1,2,3,4-tetrol) is a non-caloric polyol with a moderate sweetness of 60-80% of sucrose. Due to the high costs, it is not primarily chosen for its sweetness in confectionary field. When dissolved, erythritol exhibits a cooling effect due to its negative heat of solution. Erythritol can improve the mouth feeling and mask certain unwanted aftertastes such as astringency and the irritant effect of intense sweeteners. The majority of erythritol cannot be metabolized by the human body and is excreted unmodified into the urine without changing blood glucose and insulin levels.

Sorbitol has 50% of the relative sweetness of sugar. It has less of a tendency to cause gastric distress than mannitol. Sorbitol is highly soluble in water and is an excellent humectant. Xylitol is also called “wood sugar” has the same relative sweetness as sugar. Xylitol not only non-cariogenic, it actually prevents tooth decay. Lactitol has about 30-40% of sugar's sweetening power. Its taste and solubility profile resemble sugar. Isomalt is 45-65% as sweet as sugar and does not tend to lose its sweetness or break down during the heating process. Isomalt absorbs little water. Maltitol is 75% as sweet as sugar and provides about 2-3 kcal/g. It gives a creamy texture to the formulation.

In one embodiment, the binding composition may include hydrogenated starch hydrolysates (HSH). HSH is a form of sugar alcohol from the hydrogenation of starch. The hydrogenation process yields a mixture of several sugar alcohols. HSH are nutritive sweeteners that provide 40-90% of the sweetness of sugar.

### Polymer Stabilizer

Through extensive research, the applicant discovered that the semi-solid chewable gel compositions comprising essentially sugar alcohol or low glycemic sugars as binding agents are prone to crystallization leading to insatiability of the formulation. The applicant further discovered that certain concentration of polymer stabilizers may be used to promote the thermodynamic stability of the formulation and reducing the crystallization. In one embodiment, the semi-solid chewable gel composition is substantially sugar free and further comprises a polymer stabilizer. In one embodiment, the semi-solid chewable gel composition is substantially free of glucose, sucrose, and fructose and further comprises a polymer stabilizer.

The polymer stabilizer serves to stabilize the semi-solid chewable gel composition. In one embodiment, the polysaccharide may be water soluble.

In one embodiment, the polymer stabilizer comprises a polysaccharide of mono- or di-saccharide monomers. In one embodiment, the monomers may include glucose, fructose, mannose, galactose, arabinose, rhamnose, xylose, galacturonate, glucuronate, N-acetylgalactosamine, N-acetylglucosamine, or a combination thereof. The polysaccharide comprises from about 5 to about 50 monosaccharide monomers.

In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccahride, fructo-ligosaccharide, galactomannan oligomers, oligomers of ribose, xylose, arabinose, rhamnose, hyaluronic acid, or a combination thereof.

In one embodiment, the polymer stabilizer may include a mushroom polysaccharide. In one embodiment, the mushroom polysaccharide may derive from schizophyllum commune (Schizophyllum commue), Brazilian mushroom (Agarics blaze), Cordyceps sinensis (Cordyceps sinensis), glossy ganoderma (Ganoderma lucidum), rainbow conk (Coriolus versicolor), camphor tree sesame (Anthodia camphorate), Phellinus (Phellinus linteus), coral mushroom (Pleurotus citrinopileatus), mushroom (Lentinula edodes), Liu Songgu (Agrocybe aegerita), Hericium erinaceus (Hericium erinaceus), pleurotus eryngii (Pleurotus eryngiig), petal fine and soft (Sparrasis crispa), black fungus (Auricularia auricula), Asparagus (Flammulina velutipes) or a combination thereof. In one embodiment, the mushroom polysaccharide may include chitin, hemicellulose, α- and β-glucans, mannans, xylansand, or galactans. In one embodiment, the mushroom polysaccharides may include β-glucan polymers, with the main chain consisting of β-(1->3) linkages with some j-(1->6) branches as well as chitin, mannans, galactans, and xylans.

In one embodiment, the polymer stabilizer may include a polysaccharide derived from an herb. In one embodiment, the polysaccharide may derive from Cistanche deserticola, Astragalus membranaceus, Rubia cordifolia, Nerium indicum, Adhatoda vasica, Withania somnifera, and Glycyrrhiza glabra, aloe vera, Bletilla striata, Konjac, Goji berry, elderberry, or a combination thereof.

In one embodiment, the semi-solid composition may include from about 5% to about 15% of polymer stabilizer.

In one embodiment, the ratio of the binding composition and the polymer stabilizer is from about 8:1 to about 20:1.

The semi-solid chewable gel composition may further comprise an active pharmaceutical ingredient (API), an herbal composition, an antioxidant composition, a vitamin composition, a mineral composition, an amino acid composition, a probiotics composition, or a prebiotics composition.

Active pharmaceutical Ingredients (APIs)

Gummy can be used as an efficient delivery mechanism for an API. Firstly, gummy composition is easy to consume especially for patient populations having swallowing difficulty including without limitation pediatric and geriatric population. Second, gummy, if formulated to taste good, increases patient's compliance in taking medication. Third, when being consumed, gummy composition dissolves and coats the mucosal membrane allowing for direct absorption of APIs through a trans-mucosal process, bypassing the digestion track and avoiding first-pass effect.

In one embodiment, the API comprises a cannabinoid, an antibiotics, an anti-histamine, an anti-inflammatory agent, an fever reducer, a painkiller, a psychoactive agent, an aldosterone receptor antagonist, an angiotensin converting enzyme inhibitor, an angiotensin receptor blocker, a neprilysin inhibitor, an antiadrenergic agent, an antianginal agent, an antiarrhythmic agent, an anticholinergic chronotropic agent, an antihypertension agent, an ACE inhibitor, an angiotensin II inhibitor, an antiadrenergic agent, a beta blocker, a diuretic agent, a beta-adrenergic blocker, a calcium channel blocker, a catecholamine, an inotropic agent, a vasodilator, a renin inhibitor, a sclerosing agent, a vasopressin antagonist, a vasopressor, an anti-cholesterol agent, a statin, an agent for dyslipidemias, an antiplatelet, an anticoagulant, an antianginal agent, a corticosteroid, a beta agonist, a proton pump inhibitor, a laxative, a respiratory agent, an anti-diarrhea agent, an anti-ulcerative colitis agent, an anti-nausea agent, a renal medication, an anti-epilepsy agent, an analgesics, a muscle relaxants, an antipsychotics, a benzodiazepine, a selective serotonin reuptake inhibitor (SSRI), a diabetic agent, an antidepressant, an anti-anxiety agent, an anti-tumor agent, a stimulant, a contraceptive, a corticosteroid, an alpha blocker, a 5-alpha reductase inhibitor, a osteoporosis drug, an immune-suppressant, a PED5 inhibitor, an overactive bladder drug, an anti-gout drug, an anti-glaucoma drug, an expectorant, a cough suppressant, a sleep aid, an antifungal agent, an anti-viral agent, or a combination thereof.

In one embodiment, the API comprises abacavir (ABC), a combination of abacavir and lamivudine, abiraterone, acetazolamide, acetic acid, acetylcysteine, acetylsalicylic acid, aciclovir, adalimumab, albendazole, allopurinol, all-trans retinoid acid (ATRA), alteplase, amidotrizoate, amikacin, amiloride, amiodarone, amitriptyline, amlodipine, amodiaquine, amodiaquine, sulfadoxine with pyrimethamine, amoxicillin, amoxicillin with clavulanic acid, amphotericin B, ampicillin, anastrozole, aprepitant, arsenic trioxide, artemether, artemether with lumefantrine, artesunate, a combination of artesunate and amodiaquine, a combination of artesunate and mefloquine, a combination of artesunate and pyronaridine, tetraphosphate, ascorbic acid, asparaginase, atazanavir, a combination of atazanavir and ritonavir, atracurium, atropine, azathioprine, azithromycin, barium sulfate, BCG vaccine, beclomethasone, bedaquiline, bendamustine, benzathine benzylpenicillin, benznidazole, benzoyl peroxide, benzyl benzoate, benzylpenicillin, betamethasone, bevacizumab, bicalutamide, biperiden, bisoprolol, bleomycin, bortezomib, budesonide, a combination of budesonide and formoterol, calamine, calcium, calcium folinate, calcium gluconate, capecitabine, carbamazepine, carbetocin, carboplatin, cefalexin, cefazolin, cefixime, bupivacaine, caffeine citrate, Cefotaxime, ceftazidime, a combination of ceftazidime and avibactam and ceftriaxone, cefuroxime, charcoal, activated, chlorambucil, chloramphenicol, chlorhexidine, chlorine base compound, chloroquine, chloroxylenol, chlorpromazine, cholera vaccine, ciclosporin, ciprofloxacin, cisplatin, clarithromycin, clindamycin, clofazimine, clomifene, clomipramine, clopidogrel, clotrimazole, cloxacillin, clozapine, coagulation factor IX coagulation factor VIII, coal tar, codeine, colecalciferol, colistin, cyclizine, cyclophosphamide, cycloserine, cytarabine, dabigatran, dacarbazine, daclatasvir, dactinomycin, dapsone, darunavir, dasabuvir, dasatinib, daunorubicin, deferoxamine, delamanid, dengue vaccine, desmopressin, dexamethasone, dextran, diaphragms, diazepam, diazoxide, diethylcarbamazine, digoxin, a combination of dihydroartemisinin and piperaquine phosphate diloxanide, dimercaprol, diphtheria antitoxin, diphtheria vaccine, docetaxel, docusate sodium, dolutegravir, a combination of dolutegravir, lamivudine abd tenofovir dopamine, doxorubicin, folic acid, fomepizole, fosfomycin, gemcitabine, gentamicin, a combination of glecaprevir and pibrentasvir gliclazide doxycycline, efavirenz (EFV or EFZ), a combination of efavirenz, emtricitabine and tenofovir, a combination of efavirenz, lamivudine and tenofovir, eflornithine, a combination of emtricitabine and tenofovir, enalapril, enoxaparin, entecavir, ephedrine, epinephrine (adrenaline), ergocalciferol, ergometrine, erlotinib, erythromycin, erythropoiesis-stimulating agents, a combination of estradiol cypionate and medroxyprogesterone, acetate ethambutol, a combination of ethambutol, isoniazid, pyrazinamide, rifampicin ethambutol, isoniazid and rifampicin, ethanol, a combination of ethinylestradiol and levonorgestrel, a combination of ethinylestradiol and norethisterone, ethionamide, ethosuximide, etonogestrel-releasing implant, etoposide, fentanyl, ferrous salt, ferrous salt, folic acid, fexinidazole, filgrastim, fluconazole, flucytosine, fludarabine, fludrocortisone, fluorescein, fluorouracil, fluoxetine, a combination of isoniazid and rifampicin, isosorbide dinitrate, itraconazole, ivermectin, ketamine, lactulose, lamivudine (3TC), a combination of lamivudine, nevirapine, zidovudine, a combination of lamivudine and zidovudine, lamotrigine, latanoprost, a combination of ledipasvir and sofosbuvir, lenalidomide leuprorelin, levamisole, a combination of levodopa and carbidopa, glucagon, glucose, glucose with sodium chloride, glutaral, glyceryl trinitrate, griseofulvin, Haemophilus influenzae type b vaccine, haloperidol, halothane, heparin sodium, hydralazine. hydrochlorothiazide, hydrocortisone, hydroxocobalamin, hydroxycarbamide, hydroxychloroquine, hyoscine butylbromide, hyoscine hydrobromide, ibuprofen 2, ifosfamide, imatinib, insulin injection (soluble), intermediate-acting insulin, iodine, iohexol, ipratropium bromide, irinotecan, isoflurane, isoniazid, a combination of isoniazid, pyrazinamide and rifampicin, a combination of isoniazid, pyridoxine, sulfamethoxazole and trimethoprim, levofloxacin, levonorgestrel, levothyroxine, lidocaine, a combination of lidocaine and epinephrine (adrenaline), linezolid, a combination of lisinopril and amlodipine, a combination of lisinopril and hydrochlorothiazide, lithium carbonate, loperamide, a combination of lopinavir and ritonavir (LPV/r), loratadine, lorazepam, losartan, magnesium sulfate, mannitol, mebendazole, medroxyprogesterone acetate, mefloquine, meglumine iotroxate, melarsoprol, melphalan, mercaptopurine, meropenem, a combination of meropenem and vaborbactam, mesna, metformin, methadone, methimazole, methotrexate, methyldopa, methylprednisolone, methylthioninium chloride (methylene blue) metoclopramide, metronidazole, miconazole, midazolam, mifepristone, misoprostol, miltefosine, misoprostol, morphine, moxifloxacin, mupirocin, naloxone, natamycin, neostigmine, nevirapine (NVP), niclosamide, nicotinamide, nicotine replacement therapy (NRT) nifedipine, nifurtimox, nilotinib, nitrofurantoin, nivolumab norethisterone enantate normal immunoglobulin, oxytocin, paclitaxel, p-aminosalicylic acid, paracetamol, paromomycin, pegaspargase, penicillamine, pentamidine, permethrin, phenobarbital, phenoxymethylpenicillin, phenytoin, phytomenadione, pilocarpine, piperacillin+tazobactam, platelets, plazomicin, podophyllum resin, poliomyelitis vaccine, polymyxin B, potassium chloride, potassium ferric hexacyano-ferrate(II)-2H20 (Prussian blue), potassium iodide, potassium permanganate povidone iodine praziquantel prednisolone, primaquine, procaine benzylpenicillin procarbazinem, propofol, propranolol, propylthiouracil, prostaglandin E, protamine sulfate, pyrantel, pyrazinamide, pyridostigmine, pyridoxine, pyrimethamine, quinine, raltegravir, ranitidine, retinol, nystatin, ofloxacin, a combination of ombitasvir, paritaprevir and ritonavir, omeprazole, ondansetron, zinc sulfate, oseltamivir, oxaliplatin, oxamniquine, ribavirin 21, riboflavin, rifabutin, rifampicin, rifapentine, risperidone, ritonavir, rituximab, salbutamol, salicylic acid, selenium sulfide, senna 4, silver sulfadiazine, simvastatin, sodium calcium edetate, a combination of telmisartan and amlodipine, a combination of telmisartan and hydrochlorothiazide, tenofovir disoproxil fumarate, terbinafine, testosterone, tetracaine, tetracycline, thalidomide, thiamine, timolol, tioguanine, tiotropium, tranexamic acid, trastuzumab, triclabendazole, tropicamide, tuberculin, purified protein derivative (PPD), typhoid vaccine, ulipristal, urea, valganciclovir, valproic acid (sodium valproate), vancomycin, sodium chloride, sodium fluoride, sodium hydrogen carbonate, sodium lactate, sodium nitrite, sodium nitroprusside, sodium stibogluconate or meglumine antimoniate sodium thiosulfate, sofosbuvir, a combination of sofosbuvir and velpatasvir, spectinomycin, spironolactone, streptokinase, streptomycin, succimer, sulfadiazine, a combination of sulfadoxine and pyrimethamine, a combination of sulfamethoxazole and trimethoprim, sulfasalazine, suramin sodium, surfactant, suxamethonium, tamoxifen, varicella vaccine vecuronium verapamil vinblastine vincristine, vinorelbine, voriconazole, warfarin, zidovudine (ZDV or AZT) zinc sulfate, zoledronic acid, their derivative or combination thereof.

In one embodiment, the API comprises lidocaine, epinephrine, midazolam, morphine, bupivacaine, ketamin, propofol, acetylsalicyclic acid, ibuprofen, paracetamol (acetaminophen), codeine, fentanyl, methadone, amitriptyline, cyclizine, dexamethasone, diazepam, docusate sodium, fluoxetine, haloperidol, hyoscine butylbromide or hydrobromide, lactulose, loperamide, metoclopramide, midazolam, ondansetron, senna, hydrocortisone, loratadine, prednisolone, acetylcysteine, atropine, calcium gluconate, methylthioninium chloride, naloxone, penicillamine, Prussian blue, sodium nitrite, sodium thiosulfate, deferoxamine, dimercaprol, fomepizole, sodium calcium edetate, succimer, carbamazepine, diazepam, lamotrigine, lorazepam, magnesium sulfate, midazolam, phenobarbital, phenytoin, ethosuximide, valproic acid, albendazole, ivermectin, levamisole, mebendazole, nicodamid, praziquantel, pyrantel, albendazole, diethylcarbamazine, ivermectin, praziquantel, triclabendazole, oxamniquine, amikacin, amoxicillin, clavulanic acid, ampicillin, benzathine benzylpenicillin, benzylpenicillin, cefalexin, cefazolin, chloramphenicol, clindamycin, cloxacillin, doxycycline, gentamicin, metronidazole, nitrofurantoin, phenoxymethylpenicillin, procaine benzylpenicillin, spectinomycin, sulfamethoxazole, trimethoprim, azithromycin, cefixime, cefotaxime, ceftriaxone, cefuroxime, ciprofloxacin, clarithromycin, piperacillin, tazobactam. Vancomycin, ceftazidime, meropenem, vancomycin, ceftazidime, avibactam, colistin, Fosfomycin, linezolid, meropenem, vaborbactam, plazomycin, polymyxin B, clofazimine, dapsone, rifampicin, ethambutol, isoniazid, pyrazinamide, rifabutin, rifampicin, rifapentine, amikacin, bedaquiline, clofazimine, cycloserine, delamanid, ethionamide, levofloxacin, linezolid, meropenem, moxifloxacin, p-aminosalicylic acid, streptomycin, amphotericin B, clotrimazole, fluconazole, flucytosine, griseofulvin, itraconazole, nystatin, voriconazole, aciclovir, abacavir, lamivudine, tenofovir disoproxil fumarate, zidovudine, efavirenz, nevirapine, atazanavir, ritonavir, darunavir, lopinavir, dolutegravir, raltegravir, abacavir, dolutegravir, emtricitabine, efavirenz, nevirapine, ribavirin, valganciclovir, oseltamivir, valganiciclovir, entecavir, daclatasvir, glecaprevir, pibrentasvir, sofosbuvir, velpatasvir, ledipasvir, ombitasvir, paritaprevir, ritonavir, ribavirin, their derivative or a combination thereof.

In one embodiment, the API comprises caffeine, melatonin, nicotine, cannabidiol, acetaminophen, salicylic acid, naproxen, diphenhydramine, scopolamine, metformin, cetirizine, loratidine, chlorpheniramine, brompheniramine, alimemazine, cyprohetadine, doxylamine, hydroxyzine, promethazine, guaifenesin, codeine phosphate, dextromethorphan hydrobromide, acamprosate, baclofen, buprenorphine, naloxone, clonidine, disulfiram, methadone, naltrexone, ondansetron, bupropion, cytosine, varenicline, citalopram, clomipramine, dexopin, escitalopram, fluoxetine, fluvoxamine, imipramine, mirtazapine, paroxetine, sertraline, trazodone, amitriptyline, doxepin, mianserin, mirtazapine, trazodone, trimipramine, or a combination thereof.

In one embodiment, the API comprises an antihistamine. Example antihistamines may include acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a derivative thereof. The antihistamine composition may include one or more antihistamines.

In one embodiment, the API comprises an anti-inflammatory agent, a fever reducer, or a combination thereof. Example APIs include curcumin, gingerol, acetaminophen, aspirin, ibuprofen, naproxen, betamethasone dipropionate, betamethasone sodium phosphate, betamethasone valerate, budesonide, dexamethasone, dexamethasone sodium phosphate, sodium diclofenac, flunixin meglumine, flurbiprofen, fluticasone propionate, ketoprofen, ketorolac tromethamine, magnesium salicylate, sodium meclofenamate, mefenamic acid, methylprednisolone, methylprednisolone acetate, methylprednisolone sodium succinate, piroxicam, quinine, quinine sulfate, and sulindac.

In one embodiment, the API comprises a cough suppressant, an antitussive, an expectorant, or a combination thereof. Example APIs include guaifenesin, iodinated glycerol, ipratropium, oxitropium, tiotropium, bromhexine, carbocysteine, acetylcysteine, dextromethorphan, benzonatate, codeine, hydrocodone, methadone, butorphanol, benzonatate, ethylmorphine, oxeladin, noscapine, pipazethate, isoaminile, fominoben, clobutinol, pholcodine, doxofylline, opium, pentoxyverine, normethadone, levodropropizine, oxolamine, guacetisal, zipeprol, dropropizine, dimemorfan, clofedanol, eucalyptol, phenylpropanolamine, pidolic acid, galcanezumab, doxacurium chloride, ipeaca, cisatracurium, acetohydroxamic acid, ioxaglic acid, celiprolol, zidovudine, nisoldipine, didanosine, potassium iodide, and levopropoxyphene.

In one embodiment, the API comprises anti-diarrhea agent. Example anti-diarrhea agent comprises loperamide, bismuth subsalicylate, bismuth subgallate, Lactobacillus acidophilus, simethicone, atropine, difenoxin, diphenoxylate, attapulgite, palygorskite, crofelemer, and bisacodyl.

In one embodiment, the API comprises an anti-nausea agent. Example anti-nausea agent comprises ondansetron, granisetron, dolasetron, dexamethasone, diphenhydramine, dimenthydrainate, iorazepam, prochlorperazine, haloperidol, metoclopramide, nabilone, palonosetron chloride with netupitant, cannabinoids, hyoscine hydrobromide, cyclizine hydrochloride, promethazine, meclizine, scopolamine, perphenazine, dronabinol, marinol, aprepitant, fosaprepitant, trimethobenzamide, granisetron, dimenhydrinate, prochlorperazine edisylate, or rolapitant.

In one embodiment, the API comprises anti-depressant. Example anti-depressant agent comprises citalopram, escitalopram, fluoxetine, fluvoxamine, paroxetine, sertraline, vortioxetine, vilazodone, perphenazine, amitriptyline hydrochloride, bupropion hydrochloride, citalopram hydrobromide, clomipramine hydrochloride, trazodone hydrochloride, selegiline hydrochloride, nortriptyline hydrochloride, imipramine hydrochloride, doxepin hydrochloride, or clomipramine hydrochloride.

In one embodiment, the API comprises a pain reliever. Example pain reliver comprises acetaminophen, dilaudid, hydrocodone, oxycontin, naproxen, lavender, carisoprodol, gabapentin, capsaicin, benzocaine, ethyl 4-aminobenzoate, or tramadol hydrochloride.

In one embodiment, the API comprises an agent for treating sextual dysfunction. Example APIs include sildenafil, tadalafil, vardenafil, avanafil, mirodenafil, udenafil, iodenafil, zaprinast, icarlin, yohimbine, amantadine, cyproheptadine, neostigmine, flibanserin.

### Complexing Compositions

Complexing compositions may be useful to complex with active ingredients such as herbal extract or API therefore masking or modulating flavor profile or reducing bitterness. In one embodiment, the complexing composition comprises cyclic glucose.

Cyclodextrine can exist in several different forms. The ring structures can be highly branched and are often called cluster dextrins. Cluster dextrins have a ring structure with many branches of long chains of glucose units pendent to the ring. This has the effect of forming a helical structure. The helical structure along with the ring structure of cluster dextrins are both able to chelate small molecules. The helical structure along with the ring structure of cluster dextrin are both able to chelate the cetirizine molecule. The chelation takes place by the phenyl groups on cetirizine fitting inside the helical structure.

Cyclic dextrin is the other form of ring structured glucose. There are three major forms of cyclodextrin: alpha, beta, and gamma. Alpha cyclodextrin consists of a ring of 6 glucose units while beta has 7 glucose units in a ring and gamma has 8 glucose units in a ring. The ring structures form a crown. The inside of the crown is able to chelate small molecules.

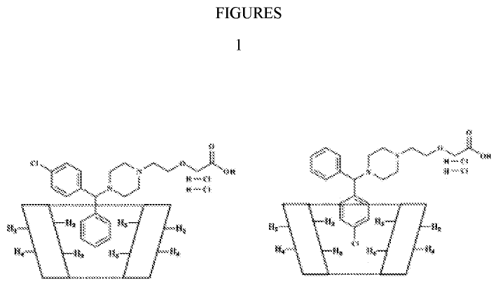

FIG. 1 shows the process of chelation of the cetirizine molecule. Either the phenyl group or the 4-chlorophenyl group fit inside the ring structure. The inside cavity of cylcodextrins is largely hydrophobic which is favorable for aromatic systems that are also hydrophobic. The formation of the chelate structure is endothermically favorable due to electrostatic interactions of the pi system of the aromatic moiety with in the hydrophobic cavity and electronic interaction with the hydrogen atoms and glycidyl ether bonds. It is these electronic interactions between these systems of the cyclodextrin and the pi system that gives the favorable heat of formation. The alpha, beta, and gamma cyclodextrins do not form the complex with cetirizine equally. Cetirizine forms more stable complex with β-cyclodextrin (K(a)=5641±358 M(−1)) than α-cyclodextrin (K(a)=1434±60 M(−1)). The association constants from ITC measurements for cetirizine-γ-cyclodextrin and cetirizine-α-cyclodextrin complexes were found to be 1200±50 and 1434 60 (−1) while the cetirizine-β-cyclodextrin complex was 5641±358 M(−1). Thus, the beta cyclodextrin has nearly four times the formation constant as the alpha and gamma cyclodextrin.

As shown in FIG. 1, the interior of the cylcodextrin is able to electronically interact with the phenyl groups of the cetirizine molecule. The phenyl group is a reverse quadrapole where the interior of the aromtic ring is very high in electron density and the exterior of the ring is electron deficent. The hydrogen atoms of the hydrophobic interior of the cyclodextrin is electronically attracted to the pi system of the aromatic ring. The hydrogen atoms of the aromatic ring are electronically attracted to the oxygen atoms of the cyclodextrin.

The coordination of cetirizine by cluster dextrins is more by chance. Cluster dextrins have a broad range of cyclic ring and helical structures. Statistically there are a few cyclic and helical structures that meet the criteria for chelation of cetirizine. The method of chelation of cetirizine by cluster dextrins is the same electronic interactions that occur as the alpha, beta, and gamma cyclodextrins.

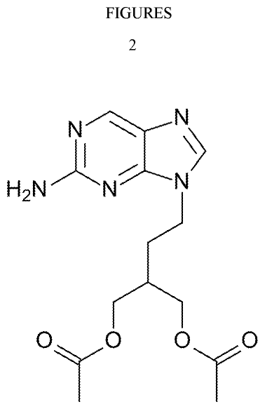

Some API's utilize nucleic acids and nucleotides as part of their structure. Famciclovir, which is a guanosine derivative, is an example of a drug with a nucleotide as part of its structure. The structure of Famciclovir is shown in FIG. 2.

Nucleic acids and structures are found in deoxyribonucleic acid (DNA) and ribonucleic acid (RNA). DNA and RNA are polymers that form helical pairs of chains. The pair of chains form through electrostatic interactions between the base pairs. The base structures on DNA consists of two purines and two pyrimidines. The purines are adenine and guanine while the two pyrimidines are cytosine and thymine. Adenine always pairs with thymine and guanine always pairs with cytosine. Uracil substitutes for thymine in RNA.

An effective method of flavor mitigation for an API that has functionality similar in structure to nucleotide bases is the addition DNA or RNA or sources rich in RNA and DNA. A chemical structure such as Famciclovir which has a guanosine structural unit will be naturally attracted and bind the cytosine bases in DNA thereby chelating the API and reducing its contribution to the overall flavor of the gummy.

Foods that are particularly high in DNA are the fruits and vegetables and their powders. Fruits and vegetables have many strands of DNA and RNA that go beyond the normal helix (diploid). The strawberry is famous for being particularly rich in DNA. Each cell in a strawberry contains 8 copies of its genetic information (octoploid), while most animal cells only contain 2 copies (diploid). Other examples of high-density DNA and/or RNA are the sweet potato (hexaploid), sugar cane (octoploid), apple (triploid), peanut (tetraploid), and kumquats (tetraploid). Any polyploidy plant material can be used as the DNA/RNA source.

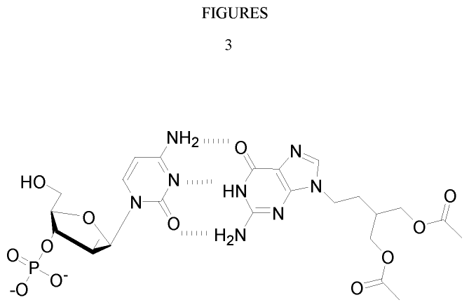

Alternatively, individual sections of DNA/RNA are added as flavor mitigators. The individual sections of DNA/RNA can be the individual base, the ribobase, or the phosphoribobase. The case of Famciclovir, as an example, cytosine, ribocytosine (cytosine riboside), or cytosine nucleotide can be utilized as flavor mitigation. FIG. 3 illustrates Famciclovir interaction with a cytosine nucleotide.

### Herbal Composition

Herbal extract or derivatives useful for the application may include flavanoids, allied phenolic compounds, polyphenolic compounds, terpenoids, alkaloids, sulphur-containing compounds, polysaccharides, flavone, flavonoids, quinone, or combinations thereof. In one embodiment, the herbal composition may comprise an adaptogen.

In one embodiment, the herbal composition comprise American ginseng (panax quinquefolius), Ashwagandha (Withania somnifera), Astragalus (Astragalus membranaceus), Cordyceps (Cordyceps militaris), Goji berry (Lycium barbarum), Eluethero root (Eleutherococcus senticosus), Jiaogulan (Gynostemma Pentaphyllum) Licorice root (Glycyrrhiza glabra), Rhodiola rosea (R. rosea), Schisandra berry/Magnolia berry (Schisandra chinensis), Tulsi/Holy basil (Ocimum sanctum), Turmeric (Curcuma longa), maca, Reishi Mushroom, Cordyceps Mushroom, Nettle Leaf Two types of phytochemicals in Zizyphus jujuba, saponins and flavonoids, trigger changes to neurotransmitters, including GABA and serotonin, which can make it easier to fall asleep and stay asleep.

At least one of the saponins in jujube, jujuboside A, helps to quiet activity in the hippocampus region of the brain. And jujube contains a flavonoid compound, spinosin, which appears to trigger sleepiness through its effects on serotonin.

Valeriana officinalis, the valerian herb, used for sleep and other medicinal purposes comes from the perennial plant, Valeriana officinalis. It's actually the root of the valerian plant that is harvested for medicinal use. Valerian primarily functions as an anxiolytic. Anxiolytics relieve anxiety and have calming, sedative effects. How does valerian lower anxiety and promote relaxation? One way, it appears, is by increasing levels of GABA (gamma-aminobutyric acid) in the brain. GABA is a chemical that our brains make naturally. GABA is what's known as an “inhibitory neurotransmitter”—it quiets the activity of the neurons of the central nervous system, which helps lower anxiety and boost feelings of relaxation and calm. GABA is an important neurochemical for sleep. Healthy levels of GABA promote and protect sound and restful sleep, and help ensure we spend the right amount of time in slow-wave sleep and REM sleep, the two deepest and most mentally and physically restorative sleep stages.

Ashwagandha, or Withania somnifera, has a long history of use in several practices, from Ayurveda to Indian and African traditional medicine. This herb is an adaptogen, a plant that may help your body cope with chronic stress, whether it's mental stress from a demanding boss or physical stress from a grueling workout. Traditional practices like Ayurveda used the root and berries of ashwagandha—also known as winter cherry or Indian ginseng—to treat a wide range of health conditions, and modem research is finding evidence to support some of these uses.

Adaptogens are herbs and plants that have been used for centuries in Ayurvedic medicine, one of the oldest holistic healing systems in the world. The adaptogen class of plants provide therapeutic benefits and help the human body deal with physical and mental stressors.

Ashwagandha can improve the quality of sleep and may help with the treatment of insomnia. Specifically, the leaves of the plant contain the compound triethylene glycol, which promotes sleep induction.

Lavender is high in essential oils that relieve stress and improve sleep. Lavender's relaxing effects are considered to be largely due to the essential oils present, whether inhaled, ingested or applied topically.

Chamomile flower provides relaxing essential oils. These oils help calm our ‘fight or flight’, sympathetic nervous system, improving sleep quality. Chamomile flower also contains an antioxidant called apigenin, said to help calm an anxious mind. What's more, chamomile tea has traditionally been used to ease digestive complaints linked to nervous conditions.

Oats have been traditionally used to recover from stress-induced nervous fatigue and to aid restful sleep.

Nutmeg, a warming spice, has a reputation for its settling and calming properties in both traditional Indonesian herbal medicine and Western herbal medicine. It's said to help you stay asleep as well as fall asleep.

Cinnamon provides a gently warming, sweet yet sugar-free flavour while helping improve blood sugar levels. Sleep deprivation increases cortisol levels, which can cause increased rates of insulin resistance; the leading cause of type 2 diabetes. Cinnamon is said to prevent this insulin resistance, as well as helping improve glucose tolerance. Cinnamon has also been said to improve indigestion, which can often end up keeping us awake at night.

### Apple Cider Vinegar

A study on humans was performed in 2009 trial with 175 people who consumed a drink containing 0, 1, or 2 tablespoons of vinegar each day. After three months, those who consumed vinegar had modest weight loss (2 to 4 pounds) and lower triglyceride levels than those who drank no vinegar. Another small study found that vinegar consumption promoted feeling fuller after eating, but that it did so by causing nausea. Neither of these studies (and none I could find in a medical literature search) specifically studied apple cider vinegar. A more recent study randomly assigned 39 study subjects to follow a restricted calorie diet with apple cider vinegar or a restricted calorie diet without apple cider vinegar for 12 weeks. While both groups lost weight, the apple cider vinegar group lost more.

### Antioxidants

Antioxidant may include astaxanthin, carotenoids, coenzyme Q10 (“CoQ10”), flavonoids, glutathione, Goji (wolfberry), hesperidin, lacto-wolfberry, lignan, lutein, lycopene, polyphenols, selenium, vitamin A, vitamin C, vitamin E, zeaxanthin, or combinations thereof.

### Vitamins

Vitamins may include vitamin A, Vitamin B1 (thiamine), Vitamin B2 (riboflavin), Vitamin B3 (niacin, inositol hexanicotinate or niacinamide), Vitamin B5 (pantothenic acid or pantothenic acid salt), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins, commonly cyanocobalamin in vitamin supplements), vitamin C, vitamin D, vitamin E, vitamin K, K1 and K2 (i.e., MK-4, MK-7), folic acid, biotin, choline, or combinations thereof.

### Minerals

Minerals may include boron, calcium, chromium, copper, iodine, iron, magnesium, manganese, molybdenum, nickel, phosphorus, potassium, selenium, silicon, tin, vanadium, zinc, amino acid chelated minerals, yeast cell wall chelated minerals, or combinations thereof.

### Amino Acids

Amino acid may include for example alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, taurine, theanine, camitine, its derivative, or combinations thereof. In one embodiment, the amino acid may be a branched-chain amino acid. In one embodiment, the amino acid may be a natural amino acid. In one embodiment, the amino acid may be a non-natural amino acid.

Taurine, or 2-aminoethanesulfonic acid, is an amino acid that is widely distributed in animal tissues. It is a major constituent of bile and can be found in the large intestine, and accounts for up to 0.1% of total human body weight. Taurine can reduce bitterness by 50% when added at a concentration of 300 mM.

### Probiotics

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects.

Probiotic may include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

The chewable composition may contain probiotic strains in an amount ranging from 10⁵ and 10¹² cfu/g. In a particular embodiment, the composition may comprise between 10⁷-10¹⁰ cfu/g.

### Prebiotics

Useful prebiotics are Lactose, Inulin, Fructo oligosacccharides, Galacto oligosaccharides and Xylo oligosaccharides. Prebiotics are naturally found plenty in certain fruits like bananas, asparagus, garlic, tomato and onion wheat. The characteristic features of ideal prebiotics are as follows. They are neither to be hydrolysed nor absorbed by mammalian enzymes or tissues. They are selectively enriched with a limited number of beneficial bacteria. The most important characteristic feature is that prebiotics can alter the intestinal micro-flora and its activities. Prebiotics can also change luminal or systemic aspects of the host defense system.

Prebiotics when combined with probiotics have many advantages. Basically, prebiotics selectively stimulate the growth of probiotics, which is dose and strain dependent. Prebiotics serve as a selective growth substrate for the probiotics strain during fermentation, during the period of storage, or during its passage through the gut.

Prebiotic may include for example acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

### Humectants

A pharmaceutically acceptable humectant can include one or a mixture of humectants, such as, for example, glycerin, sorbitol and polyethylene glycol, for the gummy composition embodiments. The humectant content can be in the range of from about of 1 weight % to about 30 weight % and such as about 2 weight % to about 25 weight %.

Humectants are low molecular weight species that give the sensation of moisture in the gummy composition. The humectants mimic water in the gummy composition and which allows for very low water levels (2-6%) in the gummy composition. The humectant can act as a moisturizing agent in the mouth. The humectant prevents the gummy composition from drying out and helps to maintain softness and shelf life. A very low vapor press is an important property of the humectant for the maintenance of softens so it does not evaporate out. Example humectants are glycerol (glycerin, 1,2,3-propantriol), propylene glycol (1,2-propandiol), aloe vera gel, glyceryl triacetate, and a-hydroxyacids (lactic acid).

### Sweeteners

Example sugar substitutes include saccharin, aspartame, sucralose and acesulfame K. In some embodiments, the sweetener used in the composition not only provides sweetness but also decreases the populations of bacteria in the mouth that lead to oral health problems.

In one embodiment, the sweetener is selected from one or more of saccharin, aspartame, cyclamate, sucralose, Stevia, mannitol, sorbitol, xylitol and similar glycols.

### Plasticizers

Plasticizers impart flexibility by lowering the glass transition temperature of the polymers. They act in gummy composition decrease brittleness and increase chewiness. Some plasticizers that can be added to the chewable are lecithin, hydrogenated vegetable oils, glycerol mono, di and tri acetate ester, lanolin, methyl ester of the fatty acids, pentaerythritol mono, di, and tri acetate ester, rice bran wax, stearic acid, sodium and potassium stearates.

### Flavoring Agents

A pharmaceutically acceptable flavoring agent can include one or a mixture of flavoring agents, such as for example bubble gum flavor, cherry flavor, grape flavor, anise oil, cassia oil, vanilla extract, vanilla creme, orange flavor, anethole, licorice, spearmint oil, phenylacetaldehyde diisobutyl acetal, and mixtures thereof, such as spearmint essential oil. Some flavoring agents can also act as sweeteners and can be use as such and include, for example, neohespiridin dehydrochalcone, xylitol, Sucralose, and mixtures thereof, such as xylitol. The flavoring agent content can be in the range of from about of 0.05 weight % to about 3 weight % and such as about 0.5 weight % to about 1 weight %.

Flavors and colors are for sensory appeal. Flavor components in gum exist in liquid, powder or micro-encapsulated forms. Liquid flavor incorporations are either water-soluble, oil-soluble, or water-dispersible emulsions. The oil-soluble flavors remain in the gum longer, resulting in longer lasting flavor sensations, because the gum base is hydrophobic and attracted to oil-based components. Conversely, water soluble flavors dissolve into saliva and as such are extract out of the gum which leads to quicker loss in flavor.

In one embodiment, the flavoring agent is a phenolic flavoring agent selected from eucalyptol, thymol, methyl salicylate, menthol, chlorothymol, phenol, wintergreen oil, spearmint oil, peppermint oil and similar essential oils, and halogenated and other derivatives thereof.

### Flavoring Modulating Agents

Taurine, or 2-aminoethanesulfonic acid, is an organic compound that is widely distributed in animal tissues. It is a major constituent of bile and can be found in the large intestine, and accounts for up to 0.1% of total human body weight. Taurine can reduce bitterness by 50% when added at a concentration of 300 mM.

### Surfactants

A pharmaceutically acceptable surfactant can include one or a mixture of surfactants, such as, for example, certain nonionic, anionic and amphoteric surfactants and can include sulfate, sulfonate and phosphate ester surfactants (e.g., alkyl sulfonates having 10 to 18 carbon atoms and sulfates of monoglycerides of fatty acids having 10 to 18 carbon atoms) as well as salts and derivatives thereof including, for example, sodium lauryl sulfate (SLS) or sodium lauryl ether sulfate (SLES) as well as anionic taurate surfactants including, for example, sodium methyl cocoyl taurate and sodium methyl oleoyl taurate, PEG caster oils, and PEG hydrogenated caster oils, including, for example, PEG-60 Hydrogenated Castor Oil Such as, the gummy composition embodiments include sodium lauryl sulfate (SLS) and sodium methyl cocoyl taurate, more such as, a mixture of sodium lauryl sulfate (SLS) and sodium methyl cocoyl taurate. Oil Such as, the gummy composition includes PEG-60 Hydrogenated Castor Oil. The surfactant content can be in the range of from about of 0.1 weight % to about 10 weight % and such as about 2 weight % to about 5 weight %.

In one embodiment, the surfactant is selected from one or more of sodium lauryl sulfate, sodium N-coco, N-methyl taurate, sodium N-lauroyl sarcosine, or a compatible food grade detergent.

### Coating Composition

The gummy pieces disclosed herein may be coated with a coating composition. In one embodiment, the coating composition may include matitol, isomalte, allulose, or a combination herein.

In one embodiment, the coating compositions may include sugar, sugar alcohol, or a combination thereof. Example sugars may include allulose, sorbose, tagatose, trehalose, and isomaltulose, or a combination thereof. Example sugar alcohols may include erythritol, sorbitol, mannitol, maltitol, isomalt, xylitol, or a combination thereof. In one embodiment, the coating composition comprises resistant starches, fibers, inulin, or a combination thereof.

### Preservatives

A pharmaceutically acceptable preservative can include one or a mixture of preservatives, such as, for example, benzoic acid, sodium benzoate, methylparaben, propylparaben, sorbic acid and potassium sorbate. These preservative agents are generally present at levels ranging from about 0.01 weight % to about 2 weight %. For the gummy composition embodiments, the example preservative is sodium benzoate.

### EXAMPLES

### Example: Low GI Gummy

Resistant Maltodextrin 130 g, Pectin, Tehalose 561 g, Isomaltulose 363 g, Sodium Citrate, Collagen, Angelica Extract, Cacao Butter, Allulose 963.3 g, Vitamin Premix 110 g, Citric Acid

To a container was added the sodium citrate, citric acid and ginkgo powder. To the container was added the water which is then heated to 200° F. To a separate container was added the pectin, isomaltulose, active mix and mixed until homogeneous. This mixture was then added to the hot water. To a separate container are added the allulose, trehalose, isomaltulose, and resistant maltodextrin. The mixture was then added mixed with water and heated on a 255° F. Cacao butter was added. Two mixtures were then combined. The flavor and color were added, and citric acid were used to adjust pH. The completed molding mixture was then poured into molds.

### Example. Sugar Free Angelica Herbal Gummy

Pectin, Sodium Citrate, Collagen, Angelica Extract, Cacao Butter, Tagatose 400 g, Resistant Maltodextrin 300 g, Allulose 1287, Cantaloupe Extract, Vitamin Premix 55.5 g, Paprika Orange 2.5 g, Cantaloupe flavor, Glycerol, Citric/Malic Acid, Monk Fruit Extract 4 g To a container was added the sodium citrate, and angelica. To the container was added the water which is then heated to 200° F. To a separate container was added the pectin, vitamin mix, cantaloupe powder, tagatose and then added to the hot water. To a separate container are added allulose, tagatose, and resistant maltodextrin. The mixture was then added to water and heated on a 255° F. Cacao butter was added followed by glycerol. Two solutions were combined and heated to Brix 83 at which time the flavor and color were added. Citric acid is used to adjust the pH. The completed molding mixture was then poured into molds.

### Example. Sugar Free Angelica Herbal Gummy

Pectin, Sodium Citrate 3 g, Collagen, Angelica Extract, Cacao Butter 50 g, Maltitol 928 g, Resistant Maltodextrin 300 g, Isomalt 759 g, Strawberry Powder, Vitamin Premix 55.5 g, Red 2.5 g, Flavor 9 g, Glycerol 3.75 g, Citric/Malic Acid 10 g, Monk Fruit Extract

To a container was added the sodium citrate, angelica and water, the mixture was then heated to 200° F. To a separate container was added the pectin, vitamin mix, strawberry powder, 200 g isomalt and mixed until homogeneous. This mixture was then added to the hot water with rapid mixing. The water becomes more viscous as the pectin dissolved. The water was brought to a gentle boil and the mixing continued for 3-5 minutes. To a separate container are added the maltitol, remaining isomalt, and resistant maltodextrin. These components are mixed until homogenous. The allulose and cyclodextrin mixture was then added to 250 g of water and heated on a 255° F. induction plate with stirring. Cacao butter was added when the mixture reached 200° F. followed by glycerol. The pectin solution was added to the allulose solution when the allulose solution began to boil with stirring. The system was then heated to Brix 83 at which time the cantaloupe flavor and citric acid and color were added. The completed molding mixture was then poured into molds.

### Example. Low GI CBD Gummy with Stevia Extract

Trehalose 470 g, Isomaltulose 313 g, Maltodextrin 300 g, Pectin, Citric Acid, Sodium Citrate, Ginkgo Extract, Stevia Extract, Hemp Oil Extract 4.9 g, Allulose 958 g, Cantaloupe Powder, gamma-Cyclodextrin 60 g, Orange color, Flavor

To a container was added the sodium citrate, citric acid and ginkgo powder. To the container was added the water which is then heated to 200° F. To a separate container was added the pectin, stevia extract, a portion of isomaltulose, cantaloupe powder, and mixed until homogeneous. This mixture was then added to the hot water with rapid mixing. The water becomes more viscous as the pectin dissolved. To a separate container are added the allulose, trehalose, remaining isomaltulose, maltodextrin and cyclodextrin. These components are mixed until homogenous. The mixture was then added to water and heated on a 255° F. Cacao butter was added when the mixture reached 200° F. The pectin solution was added to the allulose solution. The hemp oil extract was then then added to the stirring solution. The system was then heated to Brix 83 at which time the cantaloupe flavor and citric acid and color were added. The completed molding mixture was then poured into molds. Each gummy contained 12.5 mg of full spectrum hemp oil extract giving a gummy with 10 mg of CBD.

### Example. Low GI CBD Gummy with Stevia Extract

Trehalose 470 g, Isomaltulose 313 g, Maltodextrin 300 g, Pectin, Citric Acid, Sodium Citrate, Ginkgo Extract, Stevia Extract, Hemp Oil Extract 42 g, Cocoa Oil, Allulose 958 g, Pineapple Powder, gamma-Cyclodextrin 60 g, Turmeric Yellow, Flavor To a container was added the sodium citrate, citric acid and ginkgo powder. To the container was added the water, which is then heated to 200° F. To a separate container was added the pectin, stevia extract, a portion of isomaltulose, pineapple powder, and mixed until homogeneous. This mixture was then added to the hot water with rapid mixing. The water becomes more viscous as the pectin dissolved. To a separate container are added the allulose, trehalose, remaining isomaltulose, maltodextrin and cyclodextrin. The allulose and cyclodextrin mixture was then added to water and heated on a 255° F. Cacao butter was added. The pectin solution was added to the allulose solution when the allulose solution began to boil with stirring. The hemp oil extract was then then added to the stirring solution. The system was then heated to Brix 83 at which time the cantaloupe flavor and citric acid and color were added. The completed molding mixture was then poured into molds. Each gummy contained 85 mg of full spectrum hemp oil extract giving a gummy with 80 mg of CBD.

### Example. Sugar Free CBD Gummy with Stevia Extract

Tagatose 400 g, Maltodextrin 300 g, Pectin, Citric Acid, Sodium Citrate, Ginkgo Extract, Stevia Extract, Hemp Oil Extract 4.9 g, Cocoa Oil, Allulose 1341.3 g, Cantaloupe Powder, gamma-Cyclodextrin 60 g, Paprika Orange, Flavor

To a container was added the sodium citrate, citric acid and ginkgo powder. To the container was added the water which is then heated to 200° F. To a separate container was added the pectin, stevia extract, tagatose, cantaloupe powder, and mixed until homogeneous. The water becomes more viscous as the pectin dissolved. To a separate container are added the allulose, maltodextrin and cyclodextrin. These components are mixed until homogenous. The allulose and cyclodextrin mixture was then added to 250 g of water and heated on a 255° F. induction plate with stirring. Cacao butter was added when the mixture reached 200° F. The pectin solution was added to the allulose solution when the allulose solution began to boil with stirring. The hemp oil extract was then then added to the stirring solution. The system was then heated to Brix 83 at which time the cantaloupe flavor and citric acid and color were added. The completed molding mixture was then poured into molds. Each gummy contained 12.5 mg of full spectrum hemp oil extract giving a gummy with 10 mg of CBD.

### Example. CBD Gummy with Stevia Extract

Tagatose 375 g, Chicory Root Inulin 350 g, Maltodextrin 150 g, Pectin, Citric Acid, Sodium Citrate, Ginkgo Extract, Stevia Extract, Hemp Oil Extract 40 g, Cacao Butter, Allulose 1191.3 g, Pineapple Powder, gamma-Cyclodextrin, Turmeric Yellow, Flavor

To a container was added the sodium citrate, citric acid and ginkgo powder. To the container was added the water which is then heated to 200° F. To a separate container was added the pectin, stevia extract, tagatose, pineapple powder, and mixed until homogeneous. This mixture was then added to the hot water with rapid mixing. The water becomes more viscous as the pectin dissolved. The water was brought to a gentle boil and the mixing continued for 3-5 minutes. To a separate container are added the allulose, maltodextrin and cyclodextrin. These components are mixed until homogenous. The allulose and cyclodextrin mixture was then added to 250 g of water and heated on a 255° F. induction plate with stirring. Cacao butter was added when the mixture reached 200° F. The pectin solution was added to the allulose solution when the allulose solution began to boil with stirring. The hemp oil extract was then then added to the stirring solution. The system was then heated to Brix 83 at which time the pineapple flavor and citric acid and color were added. The completed molding mixture was then poured into molds. Each gummy contained 85 mg of full spectrum hemp oil extract giving a gummy with 80 mg of CBD.

### Example. Low GI Caffeinated Gummy

Sodium Citrate, Maltodextrin 214 g, gamma-Cyclodextrin 200 g, Allulose 900 g, Trehalose 500 g, Isomaltulose 300 g, Cocoa, Caffeine 44 g, Ginkgo Extract, Pectin, B vitamin mix, Strawberry Powder, Natural Flavors Strawberry, Citric Acid, Cacao Butter

In a separate container is added the allulose, trehalose, maltodextrin, cyclodextrin, cocoa, and cacao butter. These are combined and mixed until homogeneous. This is mix 1. Mix 1 is added to hot water in a separate kettle. The mixture is heated and begins to boil to create a hot syrup solution. The citric acid is added to the syrup. In a separate container is added the caffeine, ginkgo, B-mix and sodium citrate. This is mix 2. Mix 2 is added to hot water with stirring. In a separate container is added the pectin, strawberry powder, and isomaltulose are mixed until homogeneous. This is Mix 3. Mix 3 is added to the solution of Mix 2 with stirring to create a pectin solution. The pectin solution is then added to the hot syrup solution. The mixture is allowed to heat until Brix 82 at which time the flavor is added. The total mixture is then heated to Brix 83 and then added to molds.

### Example. Sugar Free Caffeinated Gummy

Sodium Citrate 1.5 g, Maltodextrin 214 g, gamma-Cyclodextrin, Allulose 1106.5 g, Tagatose 535 g, Cocoa 125 g, Caffeine 44 g, Ginkgo Extract, Pectin 65 g, B vitamin mix 8 g, Strawberry Pulp Powder, 40 g, Natural Flavors Strawberry 12 g, Citric Acid 10 g, Cacao Butter

In a separate container is added the allulose, 300 g tagatose, maltodextrin, cyclodextrin, cocoa, and cacao butter. These are combined and mixed until homogeneous. This is mix 1. Mix 1 is added to water. The kettle temperature is set to 270 F. The mixture is heated and begins to boil to create a hot syrup solution. The citric acid is added to the syrup. In a separate container is added the caffeine, ginkgo, B-mix and sodium citrate. This is mix 2. water is added to a kettle and heated to 99 C. Mix 2 is added to the water with stirring. This solution is allowed to stir for the 30 minutes. In a separate container is added the pectin, strawberry powder, and remainder of the tagatose are mixed until homogeneous. This is Mix 3. Mix 3 is added to the water solution of Mix 2 with stirring to create a pectin mixture. The solution is allowed to stir for 5-10 minutes. The pectin mixture is then added to the hot syrup. The mixture is allowed to heat until Brix 82 at which time the flavor is added. The solution is heated to Brix 83 and then added to molds.

### Example. Sugar Free Caffeinated Gummy w/Monk Fruit

Sodium Citrate 3 g, Monk Fruit Extract 15 g, gamma-Cyclodextrin, Maltodextrin 500 g, Maltitol 756.5 g, Isomlat 573.5 g, Cocoa, Caffeine 44 g, Ginkgo Extract 5 g, Pectin, B-vitamin mix 4.5 g, Orange Pulp Powder 40 g, Natural Flavors Orange 10 g, Citric Acid 12 g, Cocoa Butter 37 g

Method: In a separate container is added the maltitol, 300 g isomalt, maltodextrin, cyclodextrin, cocoa, and cacao butter. These are combined and mixed until homogeneous. This is mix 1. Mix 1 is added to a separate kettle and water is added. The mixture is heated and begins to boil to create a hot syrup solution. The citric acid is added to the syrup. In a separate container is added the caffeine, monk fruit, ginkgo, B-mix and sodium citrate. This is mix 2. water is added to a kettle and heated. Mix 2 is added to the water with stirring. This solution is allowed to stir for the 30 minutes. In a separate container is added the pectin, orange powder, and remainder of the isomalt are mixed until homogeneous. This is Mix 3. Mix 3 is added to the water solution of Mix 2 with stirring to create a pectin mixture. The solution is allowed to stir for 5-10 minutes. The pectin mixture is then added to the hot syrup. The mixture is allowed to heat until Brix 82 at which time the flavor is added. The solution is heated to Brix 83 and then added to molds.

### Example. Sugar Free Caffeinated Gummy w/Sucralose

Sodium Citrate, Sucralose 1 g, gamma-Cyclodextrin 200 g, Maltodextrin 500 g, Maltitol 756.5 g, Isomlat 573.5 g, Natural Process Cocoa 120 g, Caffeine 43 g, Ginkgo Extract, Pectin, B-vitamin mix, Orange Powder, Natural Flavors Orange, Citric Acid, Cocoa Butter

In a separate container is added the maltitol, 300 g isomalt, maltodextrin, cyclodextrin, cocoa, and cacao butter. These are combined and mixed until homogeneous. This is mix 1. Mix 1 is added to a separate kettle and water is added. The kettle temperature is set to 270 F. The mixture is heated and begins to boil to create a hot syrup solution. The citric acid is added to the syrup. In a separate container is added the caffeine, sucralose, ginkgo, B-mix and sodium citrate. This is mix 2. water is added to a kettle and heated to 99 C. Mix 2 is added to the water with stirring. This solution is allowed to stir for the 30 minutes. In a separate container is added the pectin, orange powder, and remainder of the isomalt are mixed until homogeneous. This is Mix 3. Mix 3 is added to the water solution of Mix 2 with stirring to create a pectin mixture. The solution is allowed to stir for 5-10 minutes. The pectin mixture is then added to the hot syrup. The mixture is allowed to heat until Brix 82 at which time the flavor is added. The solution is heated to Brix 83 and then added to molds.

### Example: Sugar Free Caffeine Gummy

Sodium citrate 1.5 g, maltodextrin, gamma-cyclodextrin, allulose 110 g, maltitol 550 g, cocao 100 g, caffeine 4 g, gingo biloba, pectin, multivitamin premix, strawberry powder, flavor, citric acid 10 g, cocao butter, stevia

To a kettle is added allulose, maltitol, cyclodextrin, cocoa powder, cacao butter, maltodextrin and citric acids. These components are well mixed together. To the mixture is added 300 g of cold water. The components are mixed to form a paste. The paste is slowly heated to the boiling point. The resulting syrup is heated to a brix of roughly 93. To a kettle is added caffeine, vitamin mix, ginkgo extract, followed by 500 g of water and sodium citrate. This mixture is heated to 95 C. A mixture of pectin, stevia and 200 g of allulose is created. This mixture is added to the liquids and mixed until smooth and dissolved (roughly 5 minutes). The pectin mixture is then slowly added to the hot syrup with stirring. The resulting molding mixture is heated to brix 83 and then poured into molds. The molding mixture gels into a chewable upon cooling to room temperature.

### Example: Apple Cider Vinegar Sugar Free Gummy

Apple cider vinegar 800 g, allulose 1195 g, maltitol 790 g, pectin 75 g, strawberry powder, cocao butter, malic acid, glycerol, fiber

To a kettle is added allulose, maltitol, cacao butter, polydextrose and malic/citric acids. These components are well mixed together. To the mixture is added 300 g of cold apple cider vinegar (with or without the mother). The components are mixed to form a thin paste. The paste is slowly heated to the boiling point. The resulting syrup is heated to a brix of roughly 93. To a kettle is added glycerol followed by 500 g of apple cider vinegar. This mixture is heated to 95 C. A mixture of pectin, 200 g of allulose, and strawberry powder is created. This mixture is added to the liquids and mixed until smooth and dissolved (roughly 5 minutes). The pectin mixture is then slowly added to the hot syrup with stirring. The resulting molding mixture is heated to brix 83 and then poured into molds. The molding mixture gels into a chewable upon cooling to room temperature.

### Example. Apple Cider Vinegar Gummy

Apple cider vinegar 680 g, allulose, maltitol, pectin, strawberry powder, cocao butter, citric acid, glycerol, fiber

A portion of apple cider vinegar w/mother and glycerol are combined and heated to 95° C. A portion of allulose is combined with pectin and strawberry powder and ground into a homogenous powder. The powder is added to the apple cider vinegar/glycerol with stirring and allowed to dissolve followed by continued heating for five minutes. Fiber, maltitol, cacao butter and a portion of allulose are combined and mixed together. A portion of the apple cider vinegar is added to the mixture and heated with stirring to the boiling point. Citric acid is then added to the boiling syrup and allowed to dissolve. The pectin solution is then added to the syrup with stirring. The syrup is then heated until a brix of 83 is reached. Each 7.5-gram gummy contains 100 mg of strawberry powder and 1275 mg of apple cider vinegar.

### Example: Sugar Free Herbal Gummy for Sleep Promotion

Valerian officinalis 190 g, Ziziphus jujuba see extract 150 g, melatonin 1 g, sodium citrate 5 g, citric acid 5 g, allulose 500 g, maltitol 300 g, litesse fiber 200 g, pectin, cocao butter, glycerol, flavor A portion of water and glycerol are combined and heated to 95° C. followed by the addition of sodium citrate. A portion of allulose is combined with pectin and melatonin and ground into a homogenous powder. The powder is added to the hot water/glycerol with stirring and allowed to dissolve followed by continued heating for five minutes.

Valeriana officinalis, ziziphus jujuba seed extract, fiber, maltitol, cacao butter and a portion of allulose are combined and mixed together. A portion of the water is added to the mixture and heated with stirring to the boiling point. Citric acid is then added to the boiling syrup and allowed to dissolve.

The pectin solution is then added to the syrup with stirring. The syrup is then heated until a brix of 83 is reached. Flavor is added to the syrup and then the syrup is deposited into molds and allowed to cool.

Each 7.5-gram gummy contains 500 mg Valeriana officinalis extract, 250 mg Ziziphus jujuba seed extract and 3 mg melatonin.

### Example: Sugar Free Herbal Gummy for Sleep Promotion

Valerian officinalis, ashwagandha root extract, Ziziphus jujuba see extract, melatonin, sodium citrate, citric acid, allulose, maltitol, litesse fiber, pectin, cocao butter, glycerol, flavor A portion of water and glycerol are combined and heated to 95° C. followed by the addition of sodium citrate. A portion of allulose is combined with pectin and melatonin and ground into a homogenous powder. The powder is added to the hot water/glycerol with stirring and allowed to dissolve followed by continued heating for five minutes. Valeriana officinalis, ashwagandha root extract, Ziziphus jujuba seed extract, fiber, maltitol, cacao butter and a portion of allulose are combined and mixed together. A portion of the water is added to the mixture and heated with stirring to the boiling point. Citric acid is then added to the boiling syrup and allowed to dissolve. The pectin solution is then added to the syrup with stirring. The syrup is then heated until a brix of 83 is reached. Flavor is added to the syrup and then the syrup is deposited into molds and allowed to cool. Each 7.5-gram gummy contains 300 mg Valeriana officinalis extract, 150 mg Ziziphus jujuba seed extract, 125 mg Ashwagandha root extract and 3 mg melatonin.

### Example: Sugar Free Herbal Gummy

Valeriana officinalis 100 mg, Ziziphys jujuba seed extract 100 g, melatonin 2, sodium citrate 7 g, citric acid 1 g, allulose 300 g, maltitol 500 g, polydextrose fiber 10 g, pectin 65 g, cacao butter 70 g, glycerol 60 g, flavor 8.5 g

To a kettle is added allulose, maltitol, Valeriana officinalis extract, Ziziphus jujuba seed extract, cacao butter, polydextrose and citric acids. These components are well mixed together. To the mixture is added 300 g of cold water. The components are mixed to form a thin paste. The paste is slowly heated to the boiling point. The resulting syrup is heated to a brix of roughly 93. To a kettle is added glycerol followed by 500 g of water and sodium citrate. This mixture is heated to 95 C. A mixture of pectin, 200 g of allulose, and melatonin is blended together. This mixture is added to the liquids and mixed until smooth and dissolved (roughly 5 minutes). The pectin mixture is then slowly added to the hot syrup with stirring. The resulting molding mixture is heated to brix 83 and then poured into molds. The molding mixture gels into a chewable upon cooling to room temperature.

### Example. Diphenhydramine HCl Low GI Pectin Gummy

Pectin; 1040.5 grams Allulose; 558.0 grams Trehalose, 372.0 grams Ismaltulose, 30.0 grams, Mannitol, Sodium Citrate; 35.0 grams gamma-cyclodextrin; 100 grams Maltodextrin, 10.00 grams Diphenhydramine HCl; Citric Acid; Malic Acid; 12 grams CFR Title 21 Granular Orange Flavor; 6 grams CFR Title 21 beta-Carotene Color.

In a separate container is added the allulose, trehalose, isomaltulose, maltodextrin, and mannitol. The carbohydrates are combined and mixed until homogeneous. This is mix 1. Mix 1 is added to a separate kettle and water is added. The mixture is heated and begins to boil to create a hot syrup solution. The malic and citric acids are added to the syrup. In a separate container is added the Diphenhydramine HCl, cyclodextrin and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Low GI Diphenhydramine HCl Gummy

75.0 grams pectin; 1040.5 grams Allulose; 558.0 grams Trehalose, 372.0 grams Isomaltulose, 200 grams Maltodextrin, 30.0 grams Mannitol, 3.0 grams Sodium Citrate; 80.0 grams beta-cyclodextrin, 25.00 grams Diphenhydramine HCl, Citric Acid; Malic Acid; 12 grams CFR Title 21 Granular Cherry Color; 6 grams CFR Title 21 Watermelon Red Color.

In a separate container is added allulose, trehalose, isomaltulose, gamma-cyclodextrin, maltodextrin and mannitol. They are combined and mixed until homogeneous. This is mix 1. Mix 1 is added to a separate kettle and water is added. The mixture is heated and begins to boil to create a hot syrup solution. The malic and citric acids are added to the syrup. In a separate container is added the diphenhydramine HCl, beta-cyclodextrin and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Diphenhydramine HCl Sugar Free Pectin Gummy

75.0 grams pectin; 1040.5 grams Sorbitol; 930.5 grams Xylitol, 30.0 grams, Mannitol, Sodium Citrate; 40.0 grams beta-cyclodextrin; 100 grams Maltodextrin, 10.00 grams Diphenhydramine HCl; Citric Acid; Malic Acid; 12 grams CFR Title 21 Granular Orange Flavor; 6 grams CFR Title 21 beta-Carotene Orange Color.

In a separate container is added the sorbitol, maltodextrin, cyclodextrin, xylitol and mannitol. These are combined and mixed until homogeneous. This is mix 1. Mix 1 is added to a separate kettle and water is added. The mixture is heated and begins to boil to create a hot syrup solution. The malic and citric acids are added to the syrup. In a separate container is added the diphenhydramine HCl and sodium citrate.

This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Diphenhydramine HCl Gummy

75.0 grams pectin; 1050.5 grams Maltitol; 840.5 grams Isomalt, 180 grams Maltodextrin, 30.0 grams Mannitol, Sodium Citrate; beta-cyclodextrin; 10.00 grams Diphenhydramine HCl, Citric Acid; Malic Acid; 12 grams CFR Title 21 Granular Grape Flavor; 6 grams CFR Title 21 Black Carrot Color.

In a separate container is added the maltitol, cyclodextrin, isomalt, maltodextrin and mannitol. These are combined and mixed until homogeneous. This is mix 1. Mix 1 is added to a separate kettle and water is added. The mixture is heated and begins to boil to create a hot syrup solution. The malic and citric acids are added to the syrup. In a separate container is added diphenhydramine HCl and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Diphenhydramine HCl Gummy

75.0 grams pectin; 1000 grams Maltitol; 800 grams Isomalt, 200 grams Maltodextrin, 30.0 grams Mannitol, Sodium Citrate; 80.0 grams beta-cyclodextrin; 25.00 grams Diphenhydramine HCl, 4 grams Citric Acid; Malic Acid; 12 grams CFR Title 21 Granular Cherry Color; 6 grams CFR Title 21 Watermelon Red Color. In a separate container is added the maltitol, cyclodextrin, isomalt, maltodextrin and mannitol. The three are combined and mixed until homogeneous. This is mix 1. Mix 1 is added to a separate kettle and water is added. The mixture is heated and begins to boil to create a hot syrup solution. The malic and citric acids are added to the syrup. In a separate container is added diphenhydramine HCl and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Loratadine Low GI Pectin Gummy

75 g Pectin, 1000 g Maltitol, 441 g Tehalose, 294 g Isomaltulose, 1.5 g Sodium Citrate, 40 g beta-Cyclodextrin, 30 g Mannitol, 200 g Maltodextrin, CFR 21 Orange Oil, 10 g Loratadine, 50% Citric Acid solution (in 50% glycerol/water), CFR 21 beta-Carotene Orange Color

In a separate container is added the pectin, 200 grams of maltitol, the loratadine. and sodium citrate. The components are mixed until homogeneous. This is mix 1. Water is heated to 200° F. in a kettle. To the hot water is added Mix 1. The mixture is stirred until the pectin fully swells and disperses which takes roughly 3-5 minutes. The solution is brought to a gentle boil. In a separate container is added the remaining maltitol, trehalose, isomaltulose beta-cyclodextrin, and mannitol. The components are mixed until homogeneous. This is mix 2. Mix 2 is added to a kettle followed by hot water. The kettle is heated until the contents are brought to a boil. In a separate container is added the citric acid solution, orange color, and blood orange flavor. All is mixed and warmed to 175° F. until all is dissolved. This is Mix 3. To the boiling Mix 1 solution is added Mix 2 with stirring. The mixture was heated to Brix 82 at which time Mix 3 was added dropwise with stirring. The gummy syrup was then added to silicone molds. The gummy pieces were removed from the molds to yield products containing 10 mg loratadine per piece.

### Example. Loratadine Sugar Free Pectin Gummy

75 g Pectin, 730 g Isomalt, 1.5 g Sodium Citrate, 40 g beta-Cyclodextrin, 30 g Mannitol, 1005 g Maltitol, 200 g Maltodextrin, CFR 21 Orange Oil, 10 g Loratadine, 50% Citric Acid solution (in 50% glycerol/water), CFR 21 beta-Carotene Orange Color

In a separate container is added the pectin, 200 grams of isomalt, the loratadine. and sodium citrate. The components are mixed until homogeneous. This is mix 1. Water is heated to 200° F. in a kettle. To the hot water is added Mix 1. The mixture is stirred until the pectin fully swells and disperses which takes roughly 3-5 minutes. The solution is brought to a gentle boil. In a separate container is added the remaining isomalt, maltitol, beta-cyclodextrin, and mannitol. The components are mixed until homogeneous. This is mix 2. Mix 2 is added to a kettle followed by hot water. The kettle is heated until the contents are brought to a boil. In a separate container is added the citric acid solution, orange color, and blood orange flavor. All is mixed and warmed to 175° F. until all is dissolved. This is Mix 3. To the boiling Mix 1 solution is added Mix 2 with stirring. The mixture was heated to Brix 82 at which time Mix 3 was added dropwise with stirring. The gummy syrup was then added to silicone molds. The gummy pieces were removed from the molds to yield products containing 10 mg loratadine per piece.

### Example. Tadalafil Low GI Pectin Gummy

75.0 grams pectin; 1020.5 grams Allulose; 538.0 grams Trehalose, 362.0 grams Ismaltulose, 30.0 grams, Mannitol, 3.0 grams Sodium Citrate; 35.0 grams beta-cyclodextrin; 150 grams Maltodextrin, 10.00 grams Tadalafil; Sodium Dodecyl Sulfate, Citric Acid; Malic Acid; CFR Title 21 Granular Pineapple Flavor; CFR Title 21 FD&C Yellow.

In a separate container is added the allulose, trehalose, isomaltulose, maltodextrin, and mannitol. The carbohydrates are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the tadalafil, sodium dodecyl sulfate and sodium citrate. This is mix 2. water is added to a kettle and heated to 99 C. Mix 2 is added and stirred. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Tadalafil Low GI Pectin Gummy

75.0 grams pectin; 1020.5 grams Allulose; 538.0 grams Trehalose, 362.0 grams Ismaltulose, 30.0 grams, Mannitol, 3.0 grams Sodium Citrate; 35.0 grams beta-cyclodextrin; 200 grams Maltodextrin, 6.0 grams Tadalafil; Sodium Dodecyl Sulfate, Citric Acid; Malic Acid; CFR Title 21 Granular Pineapple Flavor; CFR Title 21 FD&C Yellow.

In a separate container is added the allulose, trehalose, isomaltulose, maltodextrin, and mannitol. The carbohydrates are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the tadalafil, sodium dodecyl sulfate and sodium citrate. This is mix 2. water is added to a kettle and heated to 99 C. Mix 2 is added and stirred for 2 hours. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Tadalafil Gummy

75.0 grams pectin; 1050.5 grams Maltitol; 840.5 grams Isomalt, 180 grams Maltodextrin, 30.0 grams Mannitol, 3.0 grams Sodium Citrate; 35.0 grams beta-cyclodextrin; 10.00 grams Tadalafil, Sodium Dodecyl Sulfate, Citric Acid; Malic Acid; CFR Title 21 Granular Pineapple Flavor; CFR Title 21 FD&C Yellow Color.

In a separate container is added the maltitol, cyclodextrin, isomalt, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the tadalafil, sodium dodecyl sulfate and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Tadalafil Gummy

75.0 grams pectin; 1050.5 grams Maltitol; 840.5 grams Isomalt, 180 grams Maltodextrin, 30.0 grams Mannitol, 3.0 grams Sodium Citrate; 30.0 grams beta-cyclodextrin; 5.00 grams Tadalafil, 0.5 gram Sodium Dodecyl Sulfate, Acid; Malic Acid; 12 grams CFR Title 21 Granular Pineapple Flavor; 5 grams CFR Title 21 FD&C Yellow Color.

In a separate container is added the maltitol, cyclodextrin, isomalt, maltodextrin and mannitol. The three are combined and mixed until homogeneous. This is mix 1. Mix 1 is added to a separate kettle and water is added. The mixture is heated and begins to boil to create a hot syrup solution. The malic and citric acids are added to the syrup. In a separate container is added the tadalafil, sodium dodecyl sulfate and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 160 mg Paracetamol Low GI Gummy

Pectin 22 g, Trehalose 180 g, Isomaltulose 120 g, Sodium Citrate 1.5 g, gamma-cyclodextrin 200 g, Paracetamol 65.5 g, N-acetylglucosamine 20 g, Maltodextrin 65 g, Mannitol 30 g, Allulose 367 g, Glycerol, Citric Acid, Watermelon Red, Watermelon Flavor

In a separate container is added the trehalose, isomaltulose, allulose, maltodextrin and mannitol. The three are combined and mixed until homogeneous. This is mix 1. Mix 1 is added to a separate kettle and water is added. The mixture is heated and begins to boil to create a hot syrup solution. The malic and citric acids are added to the syrup. In a separate container is added 100 g cyclodextrin, paracetamol and sodium citrate. This is mix 2. water is added to a kettle and heated to 99 C. Mix 2 is added to the water with stirring. This solution is allowed to stir for the 30 minutes. In a separate container is added the pectin, remaining cyclodextrin and N-acetyl glucosamine. 100 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 80 mg Paracetamol Low GI Gummy

Pectin 22 g. Trehalose 180 g, Isomaltulose 120 g, Sodium Citrate 1.5 g, gamma-cyclodextrin 200 g, Paracetamol 30 g, N-acetylglucosamine 20 g, Maltodextrin 65 g, Mannitol 30 g, Allulose 367 g, Glycerol 5 g, Citric Acid 7 g, beta-Carotene Orange 2 g, Orange Flavor 4 g

In a separate container is added the trehalose, isomaltulose, allulose, maltodextrin and mannitol. The three are combined and mixed until homogeneous. This is mix 1. Mix 1 is added to a separate kettle and water is added. The mixture is heated and begins to boil to create a hot syrup solution. The malic and citric acids are added to the syrup. In a separate container is added 100 g cyclodextrin, paracetamol and sodium citrate. This is mix 2. water is added to a kettle and heated to 99 C. Mix 2 is added to the water with stirring. This solution is allowed to stir for the 30 minutes. In a separate container is added the pectin, remaining cyclodextrin and N-acetyl glucosamine. 100 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 160 mg Paracetamol Sugar Free Gummy

Pectin 22 g, Isomalt 332.1 g, Sodium Citrate 1.5 g, gamma-cyclodextrin 200 g, Paracetamol 65.5 g, N-acetylglucosamine 20 g, Maltodextrin 65 g, Mannitol 30 g, Maltitol 333.3 g, Glycerol, Citric Acid 7 g, Watermelon Red, Watermelon Flavor

In a separate container is added the maltitol, isomalt, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added 100 g cyclodextrin, paracetamol and sodium citrate. This is mix 2. water is added to a kettle and heated to 99 C. Mix 2 is added to the water with stirring. This solution is allowed to stir for the 30 minutes. In a separate container is added the pectin, remaining cyclodextrin and N-acetyl glucosamine. 100 grams from mix 1 is added to the pectin and mixed until homogeneous. This is Mix 3. Mix3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 80 mg Paracetamol Sugar Free Gummy

Pectin 22 g. Isomalt 332.1 g, Sodium Citrate 1.5 g, gamma-cyclodextrin 200 g, Paracetamol 30 g, N-acetylglucosamine 20 g, Maltodextrin, Mannitol 30 g, Maltitol 333.33 g, Glycerol 5 g, Citric Acid 7 g, beta-Carotene Orange, Orange Flavor

Method: In a separate container is added the maltitol, isomalt, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added 100 g cyclodextrin, paracetamol and sodium citrate. This is mix 2. water is added to a kettle and heated to 99 C. Mix 2 is added to the water with stirring. This solution is allowed to stir for the 30 minutes. In a separate container is added the pectin, remaining cyclodextrin and N-acetyl glucosamine. 100 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 100 mg Guaifenesin Low GI Gummy

Allulose 963 g, Trehalose 473 g, Isomaltulose 315 g, Maltodextrin 90 g, Guaifenesin USP 80 g, beta-Cyclodextrin 100 g, Pectin, Citric Acid, Sodium Citrate, Carotene Orange Color, Orange Flavor, Water

In a separate container is added allulose, trehalose, isomaltulose, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added cyclodextrin, guaifenesin and sodium citrate. This is mix 2. water is added to a kettle and heated to 99 C. Mix 2 is added to the water with stirring. This solution is allowed to stir for the 30 minutes. In a separate container is added the pectin and 300 grams from mix 1 and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 50 mg Guaifenesin Low GI Gummy

Allulose 963 g, Trehalose 473 g, Isomaltulose 315 g, Maltodextrin 150 g, Guaifenesin USP 41.5 g, gamma-Cyclodextrin 50 g, beta-Cyclodextrin 50 g, Pectin, Citric Acid, Sodium Citrate, Carotene Orange Color, Orange Flavor

In a separate container is added allulose, trehalose, isomaltulose, maltodextrin and mannitol. The three are combined and mixed until homogeneous. This is mix 1. Mix 1 is added to a separate kettle and water is added. The mixture is heated and begins to boil to create a hot syrup solution. The malic and citric acids are added to the syrup. In a separate container is added cyclodextrin, guaifenesin and sodium citrate. This is mix 2. water is added to a kettle and heated to 99 C. Mix 2 is added to the water with stirring. This solution is allowed to stir for the 30 minutes. In a separate container is added the pectin and 300 grams from mix 1 and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 100 mg Guaifenesin Sugar Free Gummy

Maltitol 962.6 g, Isomalt 787.5 g, Maltodextrin 90 g, Guaifenesin USP 80 g, beta-Cyclodextrin 100 g, Pectin, Citric Acid, Sodium Citrate, Carotene Orange Color, Orange Flavor

In a separate container is added the maltitol, isomalt, maltodextrin and mannitol. The three are combined and mixed until homogeneous. This is mix 1. Mix 1 is added to a separate kettle and water is added. mixture is heated and begins to boil to create a hot syrup solution. The malic and citric acids are added to the syrup. In a separate container is added cyclodextrin, guaifenesin and sodium citrate. This is mix 2. water is added to a kettle and heated to 99 C. Mix 2 is added to the water with stirring. This solution is allowed to stir for the 30 minutes. In a separate container is added the pectin and 300 grams from mix 1 and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 50 mg Guaifenesin Sugar Free Gummy

Maltitol 992.6 g, Isomalt 799.5 g, Maltodextrin 150 g, Guaifenesin USP 41.5 g, gamma-Cyclodextrin 50 g, beta-Cyclodextrin 50 g, Pectin, Citric Acid, Sodium Citrate, Carotene Orange Color, Orange Flavor

In a separate container is added the maltitol, isomalt, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added cyclodextrin, guaifenesin and sodium citrate. This is mix 2. water is added to a kettle and heated to 99 C. Mix 2 is added to the water with stirring. This solution is allowed to stir for the 30 minutes. In a separate container is added the pectin and 300 grams from mix 1 and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. DXM Low GI Pectin Gummy

75.0 grams pectin; 1040.5 grams Allulose; 558.0 grams Trehalose, 372.0 grams Ismaltulose, 30.0 grams, Mannitol, 3.0 grams Sodium Citrate; 35.0 grams gamma-cyclodextrin; 100 grams Maltodextrin, 10.00 grams Dextromethorphan HBr; Citric Acid; Malic Acid; Title 21 Granular Grape Flavor; CFR Title 21 Black Carrot Color.

In a separate container is added the allulose, trehalose, isomaltulose, maltodextrin, and mannitol. The carbohydrates are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the Dextromethorphan HBr, cyclodextrin and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Low GI Dextromethorphan HBr Gummy

75.0 grams pectin; 1040.5 grams Allulose; 558.0 grams Trehalose, 372.0 grams Isomaltulose, 200 grams Maltodextrin, 30.0 grams Mannitol, 3.0 grams Sodium Citrate; 40.0 grams beta-cyclodextrin; 100 grams gamma-cyclodextrin, 15.00 grams Dextromethorphan HBr, 4 grams Citric Acid; 4.0 grams Malic Acid; 12 grams CFR Title 21 Granular Cherry Color; 6 grams CFR Title 21 Watermelon Red Color.

In a separate container is added allulose, trehalose, isomaltulose, gamma-cyclodextrin, maltodextrin and mannitol. They are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the Dextromethorphan HBr, beta-cyclodextrin and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Dextromethorphan HBr Sugar Free Pectin Gummy

75.0 grams pectin; 1040.5 grams Sorbitol; 930.5 grams Xylitol, 30.0 grams, Mannitol, 3.0 grams Sodium Citrate; 35.0 grams gamma-cyclodextrin; 100 grams Maltodextrin, 10.00 grams Dextromethorphan HBr; Citric Acid; Malic Acid; CFR Title 21 Granular Orange Flavor; CFR Title 21 beta-Carotene Orange Color.

In a separate container is added the sorbitol, maltodextrin, cyclodextrin, xylitol and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the Dextromethorphan HBr and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Dextromethorphan HBr Gummy

75.0 grams pectin; 1050.5 grams Maltitol; 840.5 grams Isomalt, 180 grams Maltodextrin, 30.0 grams Mannitol, 3.0 grams Sodium Citrate; 35.0 grams beta-cyclodextrin; 10.00 grams Dextromethorphan HBr, Citric Acid; Malic Acid; CFR Title 21 Granular Grape Flavor; 6 grams CFR Title 21 Black Carrot Color.

In a separate container is added the maltitol, cyclodextrin, isomalt, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the Dextromethorphan HBr and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Dextromethorphan HBr Gummy

75.0 grams pectin; 1000 grams Maltitol; 800 grams Isomalt, 200 grams Maltodextrin, 30.0 grams Mannitol, 3.0 grams Sodium Citrate; 40.0 grams beta-cyclodextrin; 100 grams gamma-cyclodextrin, 15.00 grams Dextromethorphan HBr, Citric Acid; Malic Acid; CFR Title 21 Granular Cherry Color; CFR Title 21 Watermelon Red Color.

In a separate container is added the maltitol, cyclodextrin, isomalt, maltodextrin and mannitol. The three are combined and mixed until homogeneous. This is mix 1. Mix 1 is added to a separate kettle and water is added. The kettle temperature is set to 270 F. The mixture is heated and begins to boil to create a hot syrup solution. The malic and citric acids are added to the syrup. In a separate container is added the Dextromethorphan HBr and sodium citrate. This is mix 2. water is added to a kettle and heated to 99 C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Cetirizine Sugar Free Gelatin Gummy

Sorbitol, Mannitol, 1.3 g Cetirizine HCl, Water, Potassium Sorbate, Sodium Benzoate, Gelatin, Isomalt, Taurine

The gelatin, mannitol, cetirizine and taurine were shifted together until homogenous. The dry component mixture was added to 116.0 grams of water rapidly with rapid stirring. The dry mix quickly absorbed the water and became a rubbery mass. The rubbery mass was added to a zip lock bag and heated at 160° F. until free of foam and a clear yellow. Water was added to a container and heated to the boiling point. To the boiling water was added potassium sorbate and sodium benzoate which dissolved into the boiling water. To the boiling solution was added sorbitol and isomalt. The solution was then brought to a boil and sucrose was added. The solution was heated until 248° F. was reached and 108 grams of water removed. The solution was then cooled to 200° F. and the gelatin solution was slowly added with stirring. The mixture was stirred until homogenous. The solution was then added to silicone molds and the molds were placed in the refrigerator for 90 minutes. The gummy pieces were removed from the molds to yield products containing roughly 11 mg cetirizine per piece.

### Example. Cetirizine Gelatin Sugar Free Gummy

Sorbitol, Mannitol, 1.3 g Cetirizine HCl, Water, Potassium Sorbate, Sodium Benzoate, Gelatin, Isomalt, alpha-cyclodextrin, Taurine

The gelatin, mannitol, alpha-cyclodextrin, cetirizine and taurine were shifted together until homogenous. The dry component mixture was added to 116.0 grams of water rapidly with rapid stirring. The dry mix quickly absorbed the water and became a rubbery mass. The rubbery mass was added to a zip lock bag and heated at 160° F. until free of foam and a clear yellow. Water was added to a container and heated to the boiling point. To the boiling water was added potassium sorbate and sodium benzoate which dissolved into the boiling water. To the boiling solution was added sorbitol and isomalt. The solution was then brought to a boil and sucrose was added. The solution was heated until 248° F. was reached and 108 grams of water removed. The solution was then cooled to 200° F. and the gelatin solution was slowly added with stirring. The mixture was stirred until homogenous. The solution was then added to silicone molds and the molds were placed in the refrigerator for 90 minutes. The gummy pieces were removed from the molds to yield products containing roughly 11 mg cetirizine per piece.

### Example. Cetirizine Sugar Free Gummy

Isomalt, Mannitol, 1.3 g Cetirizine HCl, Water, Potassium Sorbate, Sodium Benzoate, Gelatin, Maltitol, beta-cyclodextrin, Taurine, Orange Flavor, Carotene color.

The gelatin, mannitol, □-cyclodextrin, cetirizine and taurine were shifted together until homogenous. The dry component mixture was added to 116.0 grams of water rapidly with rapid stirring. The dry mix quickly absorbed the water and became a rubbery mass. The rubbery mass was added to a zip lock bag and heated at 160° F. until free of foam and a clear yellow. 175 grams of water was added to a container and heated to the boiling point. To the boiling water was added potassium sorbate and sodium benzoate which dissolved into the boiling water. To the boiling solution was added maltitol and isomalt. The solution was then brought to a boil and sucrose was added. The solution was heated until 248° F. was reached and 108 grams of water removed. The solution was then cooled to 200° F. and the gelatin solution was slowly added with stirring. The mixture was stirred until homogenous at which time orange flavor and carotene color were added. The solution was then added to silicone molds and the molds were placed in the refrigerator for 90 minutes. The gummy pieces were removed from the molds to yield products containing roughly 11 mg cetirizine per piece.

### Example. Cetirizine Gelatin Sugar Reduced Ginger Gummy

Maltitol Syrup, Ginger Juice Concentrate, Sorbitol, Cetirizine Hydrochloride, Gelatin, Mannitol, Taurine, Sodium Benzoate, Potassium Sorbate, alpha-Cyclodextrin

The gelatin, cetirizine, alpha-cyclodextrin and mannitol were shifted together until homogenous. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow solution. The ginger concentrate was added to a container with sorbitol and maltitol syrup. The components were heated until reflux until a Brix level of 87.5 was reached. The components were cooled to 200° F. and the gelatin mix was added with stirring. The final Brix was 84. The solution was then added to silicone molds and the molds were placed in the refrigerator for 90 minutes. The gummy pieces were removed from the molds to yield products containing 11 mg cetirizine per piece.

### Example. Cetirizine Sugar Free Pectin Gummy with Oil

Pectin, Sorbitol, Sodium Citrate, Sucrose, Clusterdextrin, Mannitol, Coconut Oil, (Boling) Maltitol Syrup, Blood Orange Extract, Cetirizine HCl, 50% Citric Acid solution (in 50% glycerol/water), glycerol, orange color

In a separate container is added the pectin, 100 grams of sorbitol, cetirizine. and the sodium citrate. The components are mixed until homogeneous. This is mix 1. In a separate container is added the remaining sucrose, clusterdextrin, and mannitol. The components are mixed until homogeneous. This is mix 2. In a separate container is added the citric acid solution, orange color, and blood orange flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a gentle boil. To the boiling Mix 1 solution is added Mix 2 with stirring. Coconut oil is then added dropwise to the boiling mixture with stirring. The boiling glucose syrup is then added to the boiling pectin/sugar alcohol/oil mix. The mixture was heated to Brix 82 at which time Mix 3 was added dropwise with stirring. The molding mixture was then added to silicone molds. The gummy pieces were removed from the molds to yield products containing 11 mg cetirizine per piece.

### Example. Cetirizine Sugar Free Pectin Gummy

Pectin, Sorbitol, Sodium Citrate, SucroseClusterdextrin, Mannitol, (Boling) Maltitol Syrup, Blood Orange Extract, Cetirizine HCl, 50% Citric Acid solution (in 50% glycerol/water), glycerol, orange color

In a separate container is added the pectin, 100 grams of sorbitol, cetirizine. and the sodium citrate. The components are mixed until homogeneous. This is mix 1. In a separate container is added the remaining sucrose, clusterdextrin, and mannitol. The components are mixed until homogeneous. This is mix 2. In a separate container is added the citric acid solution, orange color, and blood orange flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a gentle boil. To the boiling Mix 1 solution is added Mix 2 with stirring. The boiling glucose syrup is then added to the boiling pectin/sugar alcohol/oil mix. The mixture was heated to Brix 82 at which time Mix 3 was added dropwise with stirring. The molding mixture was then added to silicone molds. The gummy pieces were removed from the molds to yield products containing 11 mg cetirizine per piece.

### Example. Agar Cetirizine Sugar Free Gummy

Ticagel Natural GC-581 B, Isomalt, Sorbitol, Mannitol, Cetirizine HCl, Citric Acid 50% in Water, orange color, Orange Natural Flavor, Glycerin

While stirring, add the Ticagel to water heated to 175° F. and mix until free of lumps. This takes roughly 10 minutes. Isomalt dissolved into water and the syrup is prewarmed to 175° F. The syrup was combined with the agar mix with stirring. Added the sorbitol with mixing for two minutes. Heat until a Brix of 80 is reached. The 50% citric acid, color, orange natural flavor, cetirizine, and glycerol were combined until homogenous. This mixture was then added to the batter. The batter was then deposited into silicone molds and cured at 98° F. and 18% relative humidity for 24 hours. The gummy pieces were removed from the molds to yield products containing 11 mg cetirizine per piece.

### Example. Kappa Carrageenan Cetirizine Sugar Free Gummy

Kappa Carrageenan, Potassium Citrate, Isomalt, Coconut oil, Sucrose, Maltitol Syrup, Cetirizine HCl, Mannitol, Blood Orange Extract In a separate container was combined the kappa carrageenan, 100 grams isomalt, and potassium citrate.

These components were mixed to water heated to 200° F. with stirring until lump free. This took approximately 10 minutes. In a separate container was added the maltitol syrup with 220 grams of isomalt and the mannitol. These were heated together until boiling. The mixture was heated until Brix 88 was reached. The mixture was cooled to 210° F. and the above solution of kappa carrageenan was slowly added with stirring. The cetirizine, blood orange extract, and 5 grams water were combined and added drop wise to the kappa/sugar mixture. The resulting molding mixture was then added to silicone molds. The gummy pieces were removed from the molds to yield products containing 11 mg cetirizine per piece.

What is claimed is:

1. A semi-solid chewable gel composition, comprising,
a gelling composition in a sufficient amount to provide a cohesive gelled product,
a binding composition comprising a sugar, a sugar alcohol, or a combination thereof, wherein the sugar comprises L-fructose, L-glucose, L-galactose, allulose, sorbose, tagatose, D-maltose (1,4-diglucose), trehalose, isomaltulose, raffinose or a combination thereof, and
a water-soluble polymer stabilizer, wherein the polymer stabilizer comprises a polymer of monosaccharide monomers selected from glucose, mannose, galactose, arabinose, rhamnose, xylose, galacturonate, glucuronate, N-acetylglucosamine, or a combination thereof, and wherein the polymer comprises from about 5 to about 500 monosaccharide monomers,
wherein the semi-solid chewable gel composition is substantially free of glucose, sucrose, and fructose.

2. The semi-solid chewable gel composition of claim 1, wherein the binding composition comprises mannitol, maltitol, isomalt, erythritol, sorbitol, xylitol, tagatose, allulose, sorbose, isomaltulose, trehalose, mannose, maltose, ribose, xylose, their acid forms or a combination thereof.

3. The semi-solid chewable gel composition of claim 1, wherein the binding composition consists substantially of mannitol, maltitol, isomalt, or a combination thereof.

4. The semi-solid chewable gel composition of claim 1, wherein the binding composition consists substantially of tagatose, allulose, trehalose, or a combination thereof.

5. The semi-solid chewable gel composition of claim 1, wherein the binding composition consists substantially of palatinose, allulose, trehalose, or a combination thereof.

6. The semi-solid chewable gel composition of claim 1, wherein the polymer of monosaccharide monomers comprise glucose monomer or mannose monomer linked through glycosidic bonds substantially free of alpha-1,4-glycosidic bond.

7. The semi-solid chewable gel composition of claim 1, wherein the polymer stabilizer comprises polydextrose, resistant starch, cellulose, maltodextrin, resistant maltodextrin, beta-glycan, soluble fiber, inulin, oligofructose, fruictooligosaccharide, mannan-oligosaccahride, mannose oligosaccharide, galacto-oligosaccahride, fructo-ligosaccharide, galactomannan oligomers, oligomers of ribose, xylose, arabinose, rhamnose, or a combination thereof.

8. The semi-solid chewable gel composition of claim 1, wherein the gelling component comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

9. The semi-solid chewable gel composition of claim 1, further comprising an active pharmaceutical ingredient (API) composition, a complexing agent, an herb composition, an antioxidant composition, a vitamin composition, a mineral composition, an amino acid composition, a probiotics composition, or a prebiotics composition.

10. The semi-solid chewable gel composition of claim 9, wherein the API composition comprises cannabinoid, an antibiotics, an anti-histamine, an anti-inflammatory agent, an fever reducer, a painkiller, a psychoactive agent, an aldosterone receptor antagonist, an angiotensin converting enzyme inhibitor, an angiotensin receptor blocker, a neprilysin inhibitor, an antiadrenergic agent, an antianginal agent, an antiarrhythmic agent, an anticholinergic chronotropic agent, an antihypertension agent, an ACE inhibitor, an angiotensin II inhibitor, an antiadrenergic agent, a beta blocker, a diuretic agent, a beta-adrenergic blocker, a calcium channel blocker, a catecholamine, an inotropic agent, a vasodilator, a renin inhibitor, a sclerosing agent, a vasopressin antagonist, a vasopressor, an anti-cholesterol agent, a statin, an agent for dyslipidemias, an antiplatelet, an anticoagulant, an antianginal agent, a corticosteroid, a beta agonist, a proton pump inhibitor, a laxative, a respiratory agent, an anti-diarrhea agent, an anti-ulcerative colitis agent, an anti-nausea agent, a renal medication, an anti-epilepsy agent, an analgesics, a muscle relaxants, an antipsychotics, a benzodiazepine, a selective serotonin reuptake inhibitor (SSRI), a diabetic agent, an antidepressant, an anti-anxiety agent, an anti-tumor agent, a stimulant, a contraceptive, a corticosteroid, an alpha blocker, a 5-alpha reductase inhibitor, a osteoporosis drug, an immune-suppressant, a PED5 inhibitor, an overactive bladder drug, an anti-gout drug, an anti-glaucoma drug, an expectorant, a cough suppressant, a sleep aid, an antifungal agent, an anti-viral agent, or a combination thereof.

11. The semi-solid chewable gel composition of claim 9, wherein the API composition comprises caffeine, melatonin, glutathione, nicotine, cannabidiol, acetaminophen, aspirin, salicylic acid, ibuprofen, naproxen, diphenhydramine, scopolamine, metaformin, cetirizine, loraditine, chlorpheniramine, brompheniramine, alimemazine, cyprohetadine, doxylamine, hydroxyzine, promethazine, guaifenesin, codeine phosphate, dextromethorphan hydrobromide, acamprosate, baclofen, buprenorphine, naloxone, clonidine, disulfiram, methadone, naltrexone, ondansetron, bupropion, cytosine, varenicline, citalopram, clomipramine, dexopin, escitalopram, fluoxetine, fluvoxamine, imipramine, mirtazapine, paroxetine, sertraline, trazodone, amitriptyline, doxepin, mianserin, mirtazapine, trazodone, trimipramine, or a combination thereof.

12. The semi-solid chewable gel composition of claim 9, wherein the API comprises an antihistamine, an anti-inflammatory, a PED5 inhibitor, a cough suppressant, an anti-depressant, an expectorant, a pain killer, or a fever reliver.

13. The semi-solid chewable gel composition of claim 12, wherein the antihistamine comprises acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramine, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a derivative thereof.

14. The semi-solid chewable gel composition of claim 9, wherein the herb composition comprises Angelica sinensis, the vitamin composition comprises Vitamin B, C and E, and the mineral composition comprise Zinc.

15. The semi-solid chewable gel composition of claim 9, wherein the herb composition comprises Cocoa and ginko biloba, the API composition comprises caffeine, the vitamin composition comprises Vitamin Bs, and the amino acid composition comprises L-theanine.

16. The semi-solid chewable gel composition of claim 9, wherein the herb composition comprises curcumin and ginger extract.

17. The semi-solid chewable gel composition of claim 9, wherein the herb composition comprises milk thistle, the antioxidant composition comprises glutathione.

18. The semi-solid chewable gel composition of claim 9, wherein the herb composition comprises valerian root extract and Ziziphus jujuba seed extract, and the API composition comprise melatonin.

19. The semi-solid chewable gel composition of claim 9, wherein the herb composition comprises valerian root extract, Ziziphus jujuba seed extract and ashwagandha extract.

20. The semi-solid chewable gel composition of claim 9, wherein the herb composition comprises apple cider vinegar.

21. The semi-solid chewable gel composition of claim 9, wherein the herb composition comprises elderberry extract and goji berry extract, the vitamin composition comprises Vitamin Bs, C, and D, and the mineral composition comprise Zinc.

22. The semi-solid chewable gel composition of claim 1, further comprising an additive selected from sweeteners, food acids, flavoring agents, coloring agents, humectants, bulking agents, fatty acids, triglycerides, plasticizers, emulsifiers, thickeners, preservatives, or and a mixture thereof.

23. The semi-solid chewable gel composition of claim 1, wherein the weight ratio of the binding composition and the polymer stabilizer is from about 10:1 to 20:1.

24. A method of making the semi-solid chewable gel composition of claim 9, comprising,
dividing the binding composition into a first binding portion and a second binding portion
combining a first mixture and water and heating to at a first elevated temperature to provide a first solution, wherein the first mixture comprising the first binding portion, the water-soluble polymer stabilizer,
combining a second mixture and water at a second elevated temperature to provide a second solution, wherein the second mixture comprises the gelling composition, the second binding portion, and optionally the complexing agent or the API composition, and wherein the second binding portion equals to at least twice the mass of the gelling composition,
combining the second solution and the first solution at a third elevated temperature to provide a third solution, wherein the third solution has a Brix number from about 80 to about 85,
adjusting pH of the third solution with a buffer salt to from about 3 to about 7.

25. A method for treating a health condition in a subject, comprising administering to the subject an effective amount of the semi-solid chewable gel composition of claim 9, wherein the subject is suffering the health condition comprising insomnia, obesity, metabolic syndrome, cardiovascular disease, erectile dysfunction, allergy, cough, inflammation, fever or pain.
